(19) United States
(12) Patent Application Publication
(10) Pub. No.: US 2019/0290564 A1
(43) Pub. Date: Sep. 26, 2019

(54) HAIR COSMETIC COMPOSITION

(71) Applicant: KAO CORPORATION, Tokyo (JP)

(72) Inventors: Jun-ichi FURUKAWA, Tokyo (JP); Shinichi TOKUNAGA, Tokyo (JP); Steven BREAKSPEAR, Darmstadt (DE); Niu JIAN, Darmstadt (DE); Bernd NOECKER, Darmstadt (DE)

(73) Assignee: KAO CORPORATION, Tokyo (JP)

(21) Appl. No.: 16/306,544

(22) PCT Filed: Jun. 2, 2017
(86) PCT No.: PCT/EP2017/063483

§ 371 Date: Nov. 30, 2018

(30) Foreign Application Priority Data
Jun. 3, 2016 (JP) 2016-112272

(51) Int. Cl. A61K 8/365; A61K 8/42; A61K 8/49; A61Q 5/04; A61K 8/34; A61Q 5/06

(52) U.S. Cl. CPC A61K 8/365; A61K 8/42; A61K 8/498; A61Q 5/04; A61K 2800/432; A61Q 5/065; A61K 2800/882; A61K 2800/884; A61K 8/347

(57) ABSTRACT

The hair cosmetic composition of the present invention, which is a one-agent type or a multi-agent type hair cosmetic composition, contains, the following components (A) to (D):
(A): a compound selected from the group consisting of glyoxylic acid, a glyoxylic acid hydrate, a glyoxylate salt, and a glyoxylamide;
(B): a phenolic compound having an electron-donating group on at least one m-position and having a hydrogen atom on at least one of the o-positions and the p-position, wherein the electron-donating group on the m-position may form, together with an adjacent carbon atom, a benzene ring optionally substituted with hydroxyl group(s);
(C): a direct dye; and
(D): water.

### FIELD OF THE INVENTION

The present invention relates to a hair cosmetic composition which can shape hair semi-permanently or permanently and dye the hair in a single treatment process.

### BACKGROUND OF THE INVENTION

In semi-permanent or permanent deforming of hair, the hair is treated with a permanent agent containing a hair-reducing agent or with a strongly alkaline treatment agent. In dyeing hair to any of various colors, the hair is treated with a hair coloring (or dyeing) agent containing an oxidation dye or a direct dye. Generally, in the case where both hair deforming and dyeing are carried out, the hair is treated with a permanent agent or the like and then with a hair dyeing agent. Such an operation requires a very long time, which reduces the turnover of clients of a beauty salon. In addition, the operation imposes a load on a client who receives the hair treatment and results in double cost. Also, when hair receives separately performed chemical treatments, the hair may be severely damaged. Thus, conventional hair treatment processes have the aforementioned problems.

To overcome such drawbacks, various techniques of directly adding a dye to a first permanent agent or a second permanent agent, for simultaneously carrying out dyeing and deforming of hair, have been adopted. Patent Document 1 discloses a keratin fiber treatment agent composition containing a disulfide direct dye and, as an alkaline agent, an inorganic or organic hydroxide having a pH of 10 to 14. Patent Document 2 discloses a method for dyeing and permanently deforming hair, the method including sequentially applying an aqueous hair dye composition containing a direct dye, an aqueous composition containing a reducing agent, a composition containing an oxidizing agent, and an aqueous composition containing a direct dye, to thereby sequentially treat keratin fiber. Patent Document 3 discloses a method for simultaneously carrying out dyeing and straightening hair, the method including treating hair with a hair treatment agent composition containing a direct dye and a carboxylic acid such as glyoxylic acid.
Patent Document 1: EP-A-2018847
Patent Document 2: EP-A-2020254
Patent Document 3: WO2014/180980

### SUMMARY OF THE INVENTION

The present invention provides a hair cosmetic composition, which is a one-agent type hair cosmetic composition composed of a single agent, or a multi-agent type hair cosmetic composition composed of multiple agents,

wherein the hair cosmetic composition comprising the following components (A) to (D):

(A): one or more species selected from the group consisting of glyoxylic acid, a glyoxylic acid hydrate, a glyoxylate salt, and a glyoxylamide;

(B): a phenolic compound having an electron-donating group on at least one m-position and having a hydrogen atom on at least one of the o-positions and the p-position, wherein the electron-donating group on the m-position may form, together with an adjacent carbon atom, a benzene ring optionally substituted with hydroxyl group(s);

(C): a direct dye; and

(D): water.

The present invention further provides a method for the treatment of hair, the method comprising the following steps (i) and (ii);

(i) a step of applying the hair cosmetic composition to hair; and

(ii) a step of heating and shaping the hair to which the hair cosmetic composition is applied.

### DETAILED DESCRIPTION OF THE INVENTION

However, the technique disclosed in Patent Document 1 essentially employs a specially designed direct dye, and the technique disclosed in Patent Document 2 requires employment of a reducing agent that can break S—S bonds of protein in hair shafts and recombine the broken bonds, and includes cumbersome steps of treatment.

The technique disclosed in Patent Document 3 gives less damage to hair and enables one-step dyeing and semi-permanently straightening of hair. However, that technique is not satisfied in some cases where a client is to receive hair deforming to a desired shape including curly hair and wavy hair, in addition to straight hair.

In view of the foregoing, the present invention is directed to a hair cosmetic composition, which gives less damage to hair; which can semi-permanently or permanently impart to hair a straight form, a wavy form, and a curly form, and simultaneously dye the deformed hair; which can semi-permanently or permanently change the shaped hair shape to another hair shape in a simple way without using a hair cosmetic composition such as a reducing agent and giving no damage to the hair; and which has high color fastness.

### Means for Solving the Problems

The present inventors have found the following. Specifically, a hair cosmetic composition containing glyoxylic acid in combination with a specific phenolic compound can semi-permanently straighten hair or impart a curly shape of a wavy shape to the hair; and can change the hair shape to any shape different therefrom only by means of heating means such as a hair iron or a curler without performing a treatment by use of a hair treatment agent such as a reducing agent, when the hair has been treated with the hair cosmetic composition. Furthermore, when a direct dye is added to the above hair cosmetic composition, simultaneous deforming and dyeing of hair can be performed. In addition, the hair cosmetic composition of the invention has excellent color fastness, which a direct dye does not possess. The present invention has been accomplished on the basis of these findings.

As used herein, the expression “deforming of hair semi-permanently or permanently” refers to the shape of hair being maintained after repeated shampooing processes (i.e., with excellent shampooing resistance). More specifically, the expression and an equivalent expression refer to, when the deformed hair is washed with a shampoo, sufficiently rinsed off with water, and naturally dried, the shape of the hair is unchanged before and after shampooing. Notably, the expression “the shape of hair is not changed” refers to no substantial change in the number of waves of wavy hair before and after shampooing, or no substantial generation of wavy hair or curled hair after shampooing straight hair.

As used herein, the term “deforming of hair (or hair deforming)” refers to a change in shape of a hair shaft via no scission or recombination of S—S bonds of a hair-component protein, and includes a change from straight hair to curly hair or the like, and a change from wavy hair, curly hair, or the like as well as naturally frizzy hair or the like to straight hair.

The hair cosmetic composition of the present invention encompasses a one-agent type hair cosmetic composition composed of a single agent, and a multi-agent type hair cosmetic composition composed of multiple agents (e.g., a 2-agent hair cosmetic composition). The multi-agent type hair cosmetic composition is categorized into a single-application composition, which is applied to hair after mixing a first agent, a second agent, and other components, and a successive-application composition, whose first agent, second agent, and the like is successively applied to hair.

As used herein, the term “the total hair cosmetic composition” refers to, in the case of a one-agent type hair cosmetic composition, the single agent forming the one-agent type hair cosmetic composition, and in the case of a single application multi-agent type hair cosmetic composition, a mixture containing all the compositions forming the multi-agent type hair cosmetic composition, at such compositional proportions that fall within the scope of the present invention, with the compositions being mixed before application of hair. In the case of a successive application multi-agent type hair cosmetic composition, which is not an actual mixture before application, “the total hair cosmetic composition” refers to a conceptual mixture containing all the compositions forming the multi-agent type hair cosmetic composition, at such compositional proportions that fall within the scope of the present invention.

### [Component (A): Glyoxylic Acid, Glyoxylic Acid Hydrate, Glyoxylate Salt, or a Glyoxylamide]

Component (A) includes any of glyoxylic acid, a glyoxylic acid hydrate, a glyoxylate salt, and a glyoxylamide. Examples of the glyoxylic acid hydrate include glyoxylic acid monohydrate. Examples of the glyoxylate salt include an alkali metal glyoxylate and an alkaline earth metal glyoxylate. Examples of the alkali metal include lithium, sodium, and potassium, and examples of the alkaline earth metal include magnesium and calcium. Examples of the glyoxylamide include N-glyoxyloylcarbocysteine and N-glyoxyloylkeratinamino acid.

From the viewpoints of enhancing a effect for deforming the shape of hair of the hair cosmetic composition of the present invention, further enhancing resistance of the hair shape against shampooing, further enhancing the re-deforming effect of the hair which has been semi-permanently deformed via heating, and further enhancing the resistance to shampooing after re-deforming of the hair, the hair cosmetic composition of the present invention preferably has a component (A) content, relative to the total composition of the hair cosmetic composition and in terms of glyoxylic acid, of 1.0 mass % or higher, more preferably 2.0 mass % or higher, still more preferably 2.5 mass % or higher, yet more preferably 3.0 mass % or higher. In addition to the aforementioned viewpoints, from a further viewpoint of suppressing irritation to the skin, the component (A) content is preferably 30 mass % or lower, more preferably 25 mass % or lower, still more preferably 20 mass % or lower, yet more preferably 15 mass % or lower, further more preferably 12 mass % or lower.

### [Component (B): Phenolic Compound Having a Specific Structure]

Component (B) is a phenolic compound having an electron-donating group on at least one m-position (preferably on two m-positions) and having a hydrogen atom on at least one of the o-positions and the p-position. The electron-donating group on the m-position may form, together with an adjacent carbon atom, a benzene ring which may be further substituted with hydroxyl group(s). From the viewpoint of permeability to hair, the molecular weight of component (B) is preferably 100 or higher, more preferably 110 or higher, and 1,000 or lower, more preferably 700 or lower, still more preferably 500 or lower. Examples of the phenolic compound of component (B) include the following components (B1), (B2), and (B3):

(B1) resorcin;
(B2) a compound represented by formula (1); and
(B3) a compound represented by formula (2).

Component (B1) is a resorcin, represented by the following formula.

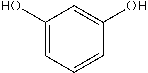

Component (B2) is a compound represented by formula (1):

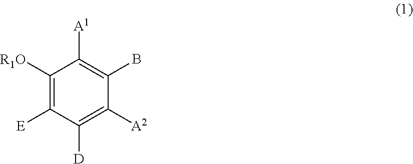

wherein:

R¹ represents a hydrogen atom or a methyl group;

A¹ and A², which may be identical to or different from each other, each represent a hydrogen atom, a C1 to C12 linear-chain or branched-chain alkyl or alkenyl group, an optionally substituted C7 to C12 aralkyl or arylalkenyl group, a C1 to C6 linear-chain or branched-chain alkoxy or alkenyloxy group, a halogen atom, or —CO—R² (wherein R² represents a C1 to C12 linear-chain or branched-chain alkyl or alkenyl group, an optionally substituted C7 to C12 aralkyl or arylalkenyl group, or an optionally substituted C6 to C12 aromatic hydrocarbon group);

B represents a hydrogen atom, a C1 to C12 linear-chain or branched-chain alkyl or alkenyl group, an optionally substituted C7 to C12 aralkyl or arylalkenyl group, or —OR³ or —COOR³ (wherein R³ represents a hydrogen atom or a C1 to C6 linear-chain or branched-chain alkyl or alkenyl group);

D represents a hydrogen atom, a hydroxyl group, a methyl group, or a C1 to C12 linear-chain or branched-chain alkoxy or alkenyloxy group; and

E represents a hydrogen atom, a hydroxyl group, a C1 to C6 linear-chain or branched-chain alkyl or alkenyl group, or a C1 to C6 linear-chain or branched-chain alkoxy or alkenyloxy group,

wherein two or three of A¹, A², B, and E each are a hydrogen atom, with each of remaining being a group which is not a sulfo group, and in the case where D is a hydrogen atom or a methyl group, A¹ and B, or A² and B form an optionally hydroxyl group-substituted benzene ring with two adjacent carbon atoms.

In the case where the aralkyl group, the arylalkenyl group, or the aromatic hydrocarbon group in represented by formula (1) have a substituent, examples of the substituent include a hydroxyl group, a C1 to C6 linear-chain or branched-chain alkyl or alkenyl group, and a C1 to C12 alkoxy group. The number of carbon atoms (Cn) of the aralkyl group, the arylalkenyl group, or the aromatic hydrocarbon group refers to the total number of the carbon atoms including those forming the substituents.

Examples of the C1 to C6 linear-chain or branched-chain alkyl or alkenyl group of R³ or E include methyl, ethyl, n-propyl, isopropyl, n-butyl, isobutyl, sec-butyl, tert-butyl, n-pentyl, isopentyl, neopentyl, 1-methylpentyl, n-hexyl, isohexyl, vinyl, allyl, butenyl, and hexenyl.

Examples of the C1 to C6 linear-chain or branched-chain alkoxy or alkenyloxy group of A¹, A², or E include groups in which the aforementioned C1 to C6 alkyl or alkenyl group to which an oxygen atom is bonded.

Examples of the C1 to C12 linear-chain or branched-chain alkyl or alkenyl group in A¹, A², R², and B include the aforementioned C1 to C6 alkyl or alkenyl groups, n-heptyl, 2,4-dimethylpentyl, 1-n-propylbutyl, n-octyl, 2-ethylhexyl, n-nonyl, 1-methylnonyl, n-decyl, 3,7-dimethyloctyl, 2-isopropyl-5-methylhexyl, n-undecyl, n-dodecyl, and decenyl.

Examples of the C1 to C12 linear-chain or branched-chain alkoxy or alkenyloxy group of D include groups formed of the aforementioned C1 to C12 alkyl or alkenyl group to which an oxygen atom is bound.

Examples of the optionally substituted C7 to C12 aralkyl or arylalkenyl group of A¹, A², R², and B include benzyl, hydroxybenzyl, dihydroxybenzyl, phenylethyl, phenylethenyl, hydroxyphenylethyl, dihydroxyphenylethyl, hydroxyphenylethenyl, dihydroxyphenylethenyl, phenylpropyl, phenylpropenyl, phenylbutyl, phenylbutenyl, phenylpentyl, phenylpentenyl, phenylhexyl, and phenylhexenyl.

Examples of the optionally substituted C6 to C12 aromatic hydrocarbon group of R² include phenyl, hydroxyphenyl, dihydroxyphenyl, trihydroxyphenyl, naphthyl, hydroxynaphthyl, and dihydroxynaphthyl.

Examples of the halogen atom of A¹ and A² include fluorine, chlorine, and bromine.

Specific examples of the compound represented by formula (1) include a resorcin derivative represented by formula (1-1), a benzophenone derivative represented by formula (1-2), and a naphthol derivative represented by formula (1-3-a) or (1-3-b), which are shown in the following.

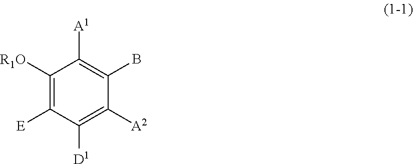

wherein R¹, A¹, A², B, and E are defined as above, and D¹ represents a hydroxyl group or a methoxy group

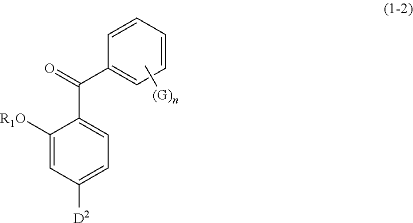

wherein R¹ has the same meaning as defined above; D² represents a hydroxyl group or a C1 to C12 alkoxy group; G represents a hydroxyl group, a C1 to C6 linear-chain or branched-chain alkyl or alkenyl group, or a C1 to C6 alkoxy group; and n is an integer of 0 to 2

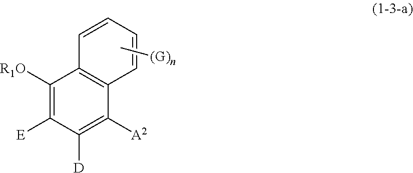

wherein R¹, A², E, D, G, and n are defined as above.

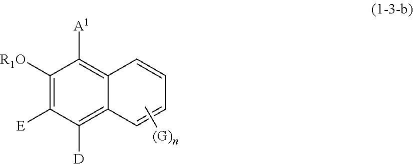

wherein R¹, A¹, E, D, G, and n are defined as above.

The compound represented by formula (1-1) is preferably any of the compounds (1-1-1) to (1-1-3).

(1-1-1) m-Dimethoxybenzene Derivative Represented by the Following Formula (1-1-1)

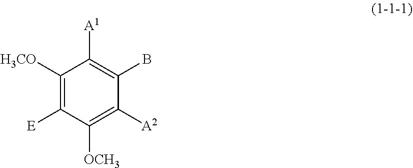

wherein A¹, A², B, and E are defined as above.

Each of A¹ and A² is preferably a hydrogen atom, a C1 to C4 linear-chain or a branched-chain alkyl or alkenyl group, more preferably a hydrogen atom.

B is preferably a hydrogen atom, a C1 to C4 alkyl or alkenyl group, an optionally substituted C7 to C10 arylalkenyl group, or a hydroxyl group, more preferably a hydrogen atom, an optionally substituted C7 to C10 arylalkenyl group, or a hydroxyl group.

E is preferably a hydrogen atom, a C1 to C4 linear-chain or branched-chain alkyl or alkenyl group, more preferably a hydrogen atom.

Examples of the compound (1-1-1) include 1,3-dimethoxybenzene, 3,5-dimethoxyphenol, 2,6-dimethoxyphenol, and 5-(hydroxyphenylethenyl)-1,3-dimethoxybenzene (trivial name: pterostilbene).

(1-1-2) m-Methoxyphenol Derivative Represented by the Following Formula (1-1-2)

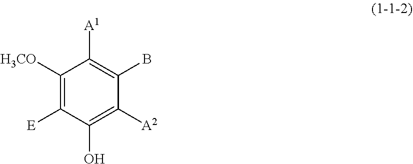

wherein A¹, A², B, and E are defined as above.

Each of A¹ and A² is preferably a hydrogen atom, a C1 to C12 linear-chain or branched-chain alkyl or alkenyl group, or an optionally substituted C7 to C12 aralkyl or arylalkenyl group, more preferably a hydrogen atom, a C1 to C6 linear-chain or branched-chain alkyl group, or an optionally substituted C7 to C10 arylalkenyl group.

B is preferably a hydrogen atom, a C1 to C12 linear-chain or branched-chain alkyl or alkenyl group, an optionally substituted C7 to C12 aralkyl or arylalkenyl group, or —OR³ (wherein R³ is a hydrogen atom or a C1 to C6 linear-chain or branched-chain alkyl or alkenyl group), more preferably a hydrogen atom, a C1 to C4 alkyl or alkenyl group, an optionally substituted C7 to C10 arylalkenyl group, or a hydroxyl group, still more preferably a hydrogen atom, an optionally substituted C7 to C10 arylalkenyl group, or a hydroxyl group.

E is preferably a hydrogen atom, a hydroxyl group, a C1 to C4 linear-chain or branched-chain alkyl or alkenyl group, or a C1 to C4 linear-chain or branched-chain alkoxy or alkenyloxy group, more preferably a hydrogen atom or a hydroxyl group.

Examples of the compound (1-1-2) include 3-methoxyphenol, 5-methoxyresorcin, 3-methoxybenzene-1,2-diol, 4-butyl-3-methoxyphenol, 3-methoxy-4-(1-phenylethyl)phenol, and 5-(4-hydroxyphenylethenyl)-1-hydroxy-3-methoxybenzene (trivial name: pinostilbene).

### (1-1-3) Resorcin Derivative Represented by the Following Formula (1-1-3)

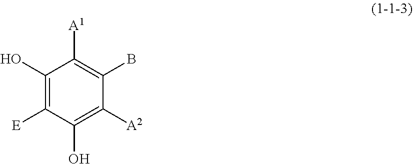

wherein A¹, A², B, and E are defined as above.

Examples of the resorcin derivative represented by formula (1-1-3) include the resorcin derivatives represented by the following formula (i) or (ii).

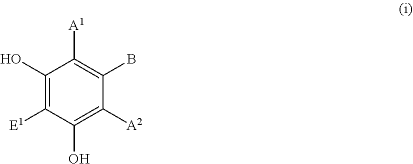

wherein A¹, A², and B are defined as above; E¹ represents a hydroxyl group, a C1 to C6 linear-chain or branched-chain alkyl or alkenyl group, or a C1 to C6 linear-chain or branched-chain alkoxy or alkenyloxy group

Each of A¹ and A² is preferably a hydrogen atom or a C1 to C12 linear-chain or branched-chain alkyl or alkenyl group, more preferably a hydrogen atom.

B is preferably a hydrogen atom, an optionally substituted C7 to C12 aralkyl or arylalkenyl group, or —OR³ (wherein R³ is a hydrogen atom or a C1 to C4 linear-chain or branched-chain alkyl or alkenyl group).

E¹ is preferably a hydroxyl group, a C1 to C4 linear-chain or branched-chain alkyl or alkenyl group, or a C1 to C4 linear-chain or branched-chain alkoxy or alkenyloxy group.

Examples of the resorcin derivative represented by formula (i) include 2-alkylresorcins such as 2-methylresorcin, 2-ethylresorcin, and 2-propylresorcin; pyrogallol; 2-alkoxyresorcins such as 2-methoxyresorcin; gallate esters such as gallic acid, methyl gallate, ethyl gallate, propyl gallate, and butyl gallate; and 5-(phenylethenyl)-2-isopropylresorcin.

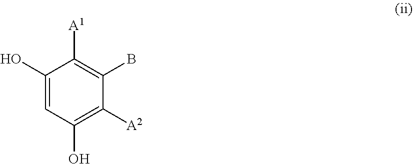

wherein A¹, A², and B are defined as above.

The resorcin derivative represented by formula (ii) is more preferably resorcin derivatives represented by formula (ii-1) or (ii-2).

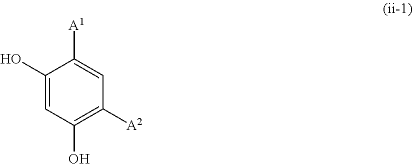

wherein A¹ and A² are defined as above.

Examples of the resorcin derivative represented by formula (ii-1) include

4-alkylresorcins such as 4-methylresorcin, 4-ethylresorcin, 4-propylresorcin, 4-isopropylresorcin, 4-butylresorcin (trivial name: rucinol), 4-isobutylresorcin, 4-sec-butylresorcin, 4-tert-butylresorcin, 4-pentylresorcin, 4-isopentylresorcin, 4-sec-pentylresorcin, 4-tert-pentylresorcin, 4-neopentylresorcin, 4-hexylresorcin, 4-isohexylresorcin, 4-heptylresorcin, 4-octylresorcin, 4-(2-ethylhexyl)resorcin, 4-nonylresorcin, 4-decylresorcin, 4-undecylresorcin, and 4-dodecylresorcin;

4-alkenylresorcins such as 4-vinylresorcin, 4-allylresorcin, 4-butenylresorcin, 4-hexenylresorcin, and 4-decenylresorcin;

4-aralkylresorcins such as 4-benzylresorcin, 4-(1-phenylethyl)resorcin (trivial name: Symwhite 377), 4-furanylethylresorcin, 4-tetrahydropyranylresorcin, 4-(2-phenylethyl)resorcin, and 4-(3-phenylpropyl)resorcin;

4-hydroxyaralkylresorcins such as 4-(4-hydroxybenzyl)resorcin, 4-(2,4-dihydroxybenzyl)resorcin, 4-(4-hydroxyphenylethyl)resorcin, and 4-(2,4-dihydroxyphenylethyl)resorcin;

4-arylalkenylresorcins such as 4-(1-phenylethenyl)resorcin and 4-(3-phenylpropenyl)resorcin;

4-hydroxyarylalkenylresorcins such as 4-(4-hydroxyphenylethenyl) resorcin and 4-(2,4-dihydroxyphenylethenyl)resorcin;

4-(1-methylnaphthyl)resorcin;

4-alkoxyresorcins such as 4-methoxyresorcin, 4-ethoxyresorcin, 4-isopropoxyresorcin, 4-propoxyresorcin, 4-butoxyresorcin, 4-sec-butoxyresorcin, 4-tert-butoxyresorcin, and 4-pentoxyresorcin;

haloresorcins such as 4-chlororesorcin and 4-bromoresorcin; 4-alkanoylresorcins such as 4-acetylresorcin, 4-propanoylresorcin, 4-butanoylresorcin, 4-pentanoylresorcin, and 4-hexanoylresorcin; and

4-arylalkanoylresorcins such as 4-phenylethanoylresorcin, 4-phenylpropanoylresorcin, 4-phenylbutanoylresorcin, 4-phenylpentanoylresorcin, 4-phenylhexanoylresorcin, 3-(hydroxyphenyl)-1-(2,4-dihydroxyphenyl)propen-1-one (trivial name: isoliquiritigenin).

Among these, from the viewpoint of obtaining more remarkable change of the shape of hair after the treatment using the hair cosmetic composition of the present invention, improving shampooing resistance of shaped hair, obtaining more remarkable change of the shape of hair during semipermanent re-deforming of the shape of hair by heating, and improving shampooing resistance of shaped hair after re-deforming of the shape of hair by means of condensate of component (A) and component (B) formed in the hair, one or more member selected from the group consisting of 4-alkylresorcin, 4-aralkylresorcin and 4-halogenated resorcin is preferable, and one or more member selected from the group consisting of 4-hexylresorcin, Rucinol, Symwhite and 4-chlororesorcin is more preferable.

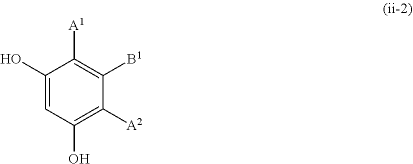

wherein A¹ and A² are defined as above; B¹ represents a C1 to C12 linear-chain or branched-chain alkyl or alkenyl group, an optionally substituted C7 to C12 aralkyl or arylalkenyl group, or —OR³ or —COOR³ (wherein R³ represents a hydrogen atom or a C1 to C6 linear-chain or branched-chain alkyl or alkenyl group).

The resorcin derivative represented by formula (ii-2) is more preferably resorcin derivatives represented by formula (ii-2-a) or (ii-2-b).

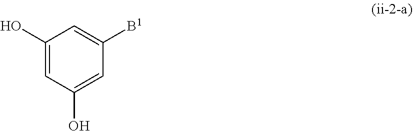

wherein B¹ has the same meaning as defined above.

Examples of the resorcin derivative represented by formula (ii-2-a) include

5-alkylresorcins such as 5-methylresorcin, 5-ethylresorcin, 5-propylresorcin, 5-isopropylresorcin, 5-butylresorcin, 5-isobutylresorcin, 5-sec-butylresorcin, 5-tert-butylresorcin, 5-pentylresorcin (trivial name: olivetol), 5-isopentylresorcin, 5-neopentylresorcin, 5-hexylresorcin, 5-isohexylresorcin, 5-heptylresorcin, 5-octylresorcin, 5-(2-ethylhexyl)resorcin, 5-nonylresorcin, 5-decylresorcin, 5-undecylresorcin, and 5-dodecylresorcin;

5-alkenylresorcins such as 5-vinylresorcin, 5-allylresorcin, 5-butenylresorcin, 5-hexenylresorcin, and 5-decenylresorcin;

phloroglucinol;

5-alkoxybenzene-1,3-diols such as 5-ethoxybenzene-1,3-diol, 5-propoxybenzene-1,3-diol, and 5-butoxybenzene-1,3-diol;

3,5-dihydroxybenzoic acid;

3,5-dihydroxybenzoate esters such as methyl 3,5-dihydroxybenzoate, ethyl 3,5-dihydroxybenzoate, propyl 3,5-dihydroxybenzoate, butyl 3,5-dihydroxybenzoate, pentyl 3,5-dihydroxybenzoate, and hexyl 3,5-dihydroxybenzoate;

5-aralkylresorcins such as 5-benzylresorcin, 5-(1-phenylethyl)resorcin, 5-(2-phenylethyl)resorcin, and 5-(phenylpropyl) resorcin;

5-hydroxyaralkylresorcins such as 5-(4-hydroxybenzyl)resorcin, 5-(2,4-dihydroxybenzyl)resorcin, 5-(hydroxyphenylethyl)resorcin (trivial name: dihydroresveratrol), and 5-(2,4-dihydroxyphenylethyl) resorcin;

5-arylalkenylresorcins such as 5-(phenylethenyl)resorcin (trivial name: pinosylvin) and 5-(phenylpropenyl)resorcin; and

5-hydroxyarylalkenylresorcins such as 5-(4-hydroxyphenylethenyl)resorcin (trivial name: resveratrol), 5-(4-methoxyphenylethenyl)resorcin (trivial name: 4-methoxyresveratrol), 5-(2,4-dihydroxyphenylethenyl)resorcin (trivial name: oxyresveratrol), 5-(2-methoxy-4-hydroxyphenylethenyl)resorcin (trivial name: gnetucleistol D), 5-(3,4-dimethoxyphenylethenyl)resorcin (trivial name: gnetucleistol E), 5-(3-hydroxy-4-methoxyphenylethenyl)resorcin (trivial name: rhapontigenin), 5-(4-hydroxy-3-methoxyphenylethenyl)resorcin (trivial name: isorhapontigenin), and 5-(dihydroxyphenylethenyl)resorcin (trivial name: piceatannol).

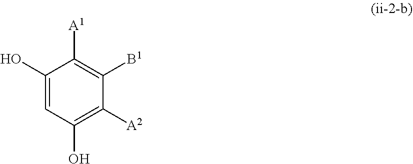

wherein A¹, A², and B¹ are defined as above.

Each of A¹ and A² is preferably a hydrogen atom, a C1 to C4 linear-chain or branched-chain alkyl or alkenyl group, or a C1 to C4 alkoxy or alkenyloxy group.

Examples of the resorcin derivative represented by formula (ii-2-b) include 2-alkylbenzene-1,3,5-triols such as 2-methylbenzene-1,3,5-triol, 2-ethylbenzene-1,3,5-triol, 2-propylbenzene-1,3,5-triol, 2-butylbenzene-1,3,5-triol, 2-hexylbenzene-1,3,5-triol, 2-octylbenzene-1,3,5-triol, and 2-dodecylbenzene-1,3,5-triol;

2-aralkyl-1,3,5-triols such as 2-benzylbenzene-1,3,5-triol, 2-(phenylethyl)benzene-1,3,5-triol, and 2-(phenylpropyl)benzene-1,3,5-triol;

2,4,6-trihydroxyphenylaralkylketone such as 2-acetylbenzene-1,3,5-triol, 2-propanoylbenzene-1,3,5-triol, 2-butanoylbenzene-1,3,5-triol, 2-phenylethanoylbenzene-1,3,5-triol, 2-hydroxyphenyl-1-(benzene-2,4,6-triol)ethan-1-one, 3-hydroxyphenyl-1-(benzene-2,4,6-triol)propan-1-one (trivial name: phloretin), 4-hydroxyphenyl-1-(benzene-2,4,6-triol)butan-1-one, 2-benzoylbenzene-1,3,5-triol, 2-(hydroxybenzoyl)benzene-1,3,5-triol, 2-(3,5-dihydroxybenzoyl)benzene-1,3,5-triol, and 2-(2,4-dihydroxybenzoyl)benzene-1,3,5-triol; and

3,5-dihydroxybenzoate esters such as 3,5-dihydroxy-2-methylbenzoic acid, methyl 3,5-dihydroxy-2-methylbenzoate, 3,5-dihydroxy-2-ethylbenzoic acid, methyl 3,5-dihydroxy-2-ethylbenzoate, 3,5-dihydroxy-2-propylbenzoic acid, methyl 3,5-dihydroxy-2-propylbenzoate, 3,5-dihydroxy-2-butylbenzoic acid, and methyl 3,5-dihydroxy-2-butylbenzoate.

Examples of the benzophenone derivative represented by formula (1-2) include 4-benzoylresorcin (trivial name: Benzophenone-1), 4-(hydroxybenzoyl)resorcin, 4-(dihydroxybenzoyl)resorcin, 4-(2,4-dihydroxybenzoyl)resorcin (trivial name: Benzophenone-2), 4-(methylbenzoyl)resorcin, 4-(ethylbenzoyl)resorcin, 4-(dimethylbenzoyl)resorcin, 4-(diethylbenzoyl)resorcin, 4-naphthoylresorcin, 2-hydroxy-4-methoxybenzophenone (trivial name: Benzophenone-3), 2,2′-dihydroxy-4,4′-dimethoxybenzophenone (trivial name: Benzophenone-6), 2,2′-dihydroxy-4-methoxybenzophenone (trivial name: Benzophenone-8), 2-hydroxy-4-methoxy-4′-methylbenzophenone (trivial name: Benzophenone-10), and 2-hydroxy-4-octyloxybenzophenone (trivial name: Benzophenone-12).

Among the naphthol derivatives represented by formula (1-3-a) or (1-3-b), preferred are those represented by formula (1-3-a) or (1-3-b) in which R¹ is a hydrogen atom or a C1 to C4 alkyl or alkenyl group, with a hydrogen atom being more preferred.

Also, in the above naphthol derivatives, each of A¹ and A² is preferably a hydrogen atom, a hydroxyl group, a C1 to C4 linear-chain or branched-chain alkyl group, or a C1 to C4 alkoxy group, more preferably a hydrogen atom or a hydroxyl group.

Also, in the above naphthol derivatives, D is preferably a hydrogen atom, a hydroxyl group, a C1 to C4 linear-chain or branched-chain alkyl group, or a C1 to C4 alkoxy group.

Also, in the above naphthol derivatives, E is preferably a hydrogen atom, a hydroxyl group, a C1 to C4 alkyl group, or a C1 to C4 alkoxy group.

Examples of the above compounds include 1-naphthol, 2-naphthol, 3-methylnaphthalen-1-ol, naphthalene-1,4-diol, naphthalene-1,5-diol, and naphthalene-1,8-diol.

Among the compounds represented by formula (1), preferred are m-dimethoxybenzene derivatives represented by formula (1-1-1), resorcin derivatives represented by formula (1-1-3), benzophenone derivatives represented by formula (1-2), and naphthol derivative represented by formula (1-3-a) or (1-3-b). More preferred compounds are 2-methylresorcin, 4-chlororesorcin, 4-alkylresorcin, 4-aralkylresorcin, 4-acylated resorcin, 5-alkylresorcin, 5-aralkylresorcin, 5-hydroxyarylalkenylresorcin, 2,4,6-trihydroxyphenylaralkylketone, gallic acid, and a gallate ester. Still more preferred compounds are 4-butylresorcin (trivial name: rucinol), 4-hexylresorcin, 4-(1-phenylethyl)resorcin (trivial name: Symwhite 377), 4-furanylethylresorcin, 4-tetrahydropyranylresorcin, 5-(hydroxyphenylethenyl)resorcin (trivial name: resveratrol), 3-hydroxyphenyl-1-(benzene-2,4,6-triol)propan-1-one (trivial name: phloretin), 4-(2,4-dihydroxybenzoyl)resorcin (trivial name: Benzophenone-2), 5-(hydroxyphenylethenyl)-1,3-dimethoxybenzene (trivial name: pterostilbene), and 1-naphthol. Yet more preferred compounds are 2-methylresorcin, 4-chlororesorcin, 1-naphthol, 4-n-butylresorcin, 4-(1-phenylethyl)resorcin, 5-(hydroxyphenylethenyl)resorcin, 3-hydroxyphenyl-1-(benzene-2,4,6-triol)propan-1-one, and 4-(2,4-dihydroxybenzoyl)resorcin.

In addition, among the compound represented by general formula (1), obtaining more remarkable change of the shape of hair after the treatment using the hair cosmetic composition of the present invention, as well as improving restoration of damaged hair damaged by chemical treatment, by means of condensate of component (A) and component (B) formed in the hair, one or more member selected from the group consisting of m-dimethoxybenzene derivatives represented by general formula (1-1-1), resorcin derivatives represented by general formula (1-1-3), benzophenone derivatives represented by general formula (1-2), and naphthol derivatives represented by general formula (1-3-a) or (1-3-b) is preferable.

Furthermore, one or more member selected from the group consisting of 2-alkylresorcin, 4-alkylresorcin, 4-aralkyl resorcin, 4-halogenated resorcin, 5-hydroxyarylalkenylresorcin, 2,4,6-trihydroxyphenylaralkylketone, benzophenone derivative, naphthol, 4-acylated resorcin, 5-alkylresorcin, 5-aralkylresorcin, gallic acid and gallic acid ester is preferable.

Furthermore, one or more member selected from the group consisting of 2-methylresorcin, 4-butylresorcin (trivial name: Rucinol), 4-hexylresorcin, 4-(1-phenylethyl)resorcin (trivial name: Symwhite377), 4-chlororesorcin, 5-(hydroxyphenylethenyl)resorcin (trivial name: resveratrol), 5-(hydroxyphenylethenyl)-1,3-dimethoxybenzene (trivial name: Pterostilbene), 3-hydroxyphenyl-1-(benzene-2,4,6-triol)propane-1-on (trivial name: Phloretin), 4-(2,4-dihydroxybenzoyl)resorcin (trivial name: Benzophenone-2) and 1-naphthol is preferable.

The compound represented by formula (1) preferably has a molecular weight of 120 or higher, and from the viewpoint of permeability to hair, 1,000 or lower, more preferably 500 or lower, still more preferably 300 or lower.

Component (B3) is a compound represented by the following formula (2).

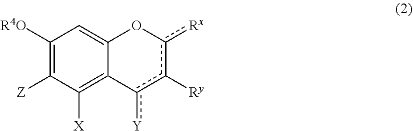

wherein,

R⁴ represents a hydrogen atom or a methyl group;

X represents a hydrogen atom, a hydroxyl group, or a methoxy group;

Y represents a hydrogen atom, an oxygen atom, a hydroxyl group, or a methoxy group;

Z represents a hydrogen atom or a C1 to C5 linear-chain or branched-chain alkyl or alkenyl group;

R^{x} represents a hydrogen atom, an oxygen atom, a hydroxyl group, a methoxy group, or an aromatic hydrocarbon group which may be substituted with 1 to 3 hydroxyl groups or methoxy groups and which optionally forms a condensed ring with 1,3-dioxolane;

R^{y} represents a hydrogen atom, a hydroxyl group, a methoxy group, an aromatic hydrocarbon group which may be substituted with 1 to 3 hydroxyl groups or methoxy groups and which optionally forms a condensed ring with 1,3-dioxolane, or an arylcarbonyloxy or aralkylcarbonyloxy group which may be substituted with 1 to 3 hydroxyl groups or methoxy groups;

a dotted line portion may be a double bond;

each of the combinations of the dotted line and the solid line being adjacent to R^{x} or Y denotes a double bond in the case where R^{x} or Y is an oxygen atom, and denotes a single bond in the other cases; and

Z represents a C1 to C5 linear-chain or branched-chain alkyl or alkenyl group, only when R^{x} or R^{y} is an o,p-dihydroxyaromatic hydrocarbon group, and represents a hydrogen atom in the other cases

Examples of the C1 to C5 linear-chain or branched-chain alkyl or alkenyl group of Z include methyl, ethyl, n-propyl, isopropyl, n-butyl, isobutyl, sec-butyl, tert-butyl, n-pentyl, isopentyl, sec-pentyl, tert-pentyl, neopentyl, 1-methylpentyl, vinyl, allyl, and butenyl.

Examples of the aromatic hydrocarbon group of R^{x} or R^{y} include phenyl and naphthyl. Examples of the aromatic hydrocarbon group which forms a condensed ring with 1,3-dioxolane include 1,3-benzodioxol-5-yl group.

Examples of the arylcarbonyloxy group of R^{y} include a benzoyloxy group, and examples of the aralkylcarbonyloxy group of R^{y} include a benzylcarbonyloxy group, a phenylethylcarbonyloxy group, a phenylpropylcarbonyloxy group, and a phenylbutylcarbonyloxy group.

Specific examples of the compound represented by formula (2) include compounds represented by the following formulas (2-1) to (2-5).

### (2-1) Flavanols Represented by the Following Formula (2-1)

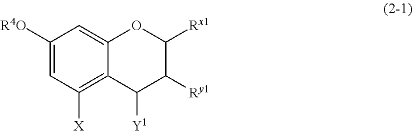

wherein

R⁴ and X are defined as above;

Y¹ represents a hydrogen atom, a hydroxyl group, or a methoxy group;

R^{x1} represents an aromatic hydrocarbon group which may be substituted with 1 to 3 hydroxyl groups or methoxy groups and which optionally forms a condensed ring with 1,3-dioxolane; and

R^{y1} represents a hydrogen atom, a hydroxyl group, a methoxy group, an aromatic hydrocarbon group which may be substituted with 1 to 3 hydroxyl groups or methoxy groups and which optionally forms a condensed ring with 1,3-dioxolane, or an arylcarbonyloxy or aralkylcarbonyloxy group which may be substituted with 1 to 3 hydroxyl groups or methoxy groups

### (2-2) Flavanones or Flavanonol Represented by the Following Formula (2-2)

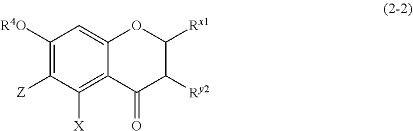

wherein R⁴, X, Z, and R^{x1} are defined as above, and R^{y2} represents a hydrogen atom, a hydroxyl group, or a methoxy group; and

Z represents a C1 to C5 linear-chain or branched-chain alkyl or alkenyl group, only when R^{x1} is an o,p-dihydroxyaromatic hydrocarbon group, and represents a hydrogen atom in the other cases

### (2-3) Flavanones or Flavanonol Represented by the Following Formula (2-3)

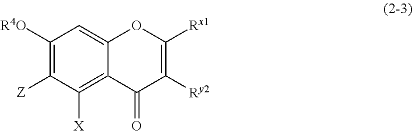

wherein R⁴, X, Z, R^{x1,} and R^{y2} are defined as above. Z is a C1 to C5 linear-chain or branched-chain alkyl or alkenyl group, only when R^{x1} is an o,p-dihydroxyaromatic hydrocarbon group. In the other cases, Z is a hydrogen atom.

### (2-4) Isoflavons and Isoflavans Represented by the Following Formula (2-4)

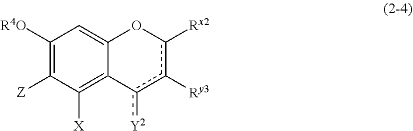

wherein

R⁴, X, Z, and the dotted line portion are defined as above;

Y² represents a hydrogen atom or an oxygen atom;

R^{x2} represents a hydrogen atom, a hydroxyl group, or a methoxy group;

R^{y3} represents an aromatic hydrocarbon group which may be substituted with 1 to 3 hydroxyl groups or methoxy groups and which optionally forms a condensed ring with 1,3-dioxolane;

the combination of the dotted line and the solid line being adjacent to Y² denotes a double bond in the case where Y² is an oxygen atom, and denotes a single bond in the other cases; and

Z represents a C1 to C5 linear-chain or branched-chain alkyl or alkenyl group, only when R^{y3} is an o,p-dihydroxyaromatic hydrocarbon group, and represents a hydrogen atom in the other cases

### (2-5) Coumarins Represented by Formula (2-5)

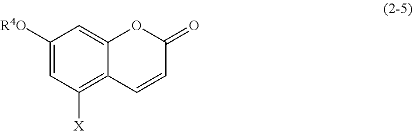

wherein R⁴ and X are defined as above.

The compound (2-1) is preferably compound represented by the following formula (2-1-A) to (2-1-C).

### (2-1-A) Flavan-3-ols Represented by the Following Formula (2-1-A)

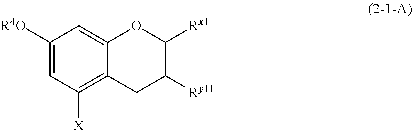

wherein R⁴, X, and R^{x1} are defined as above; R^{y11} represents a hydroxyl group, a methoxy group, an aromatic hydrocarbon group which may be substituted with 1 to 3 hydroxyl groups or methoxy groups and which optionally forms a condensed ring with 1,3-dioxolane, or an arylcarbonyloxy or aralkylcarbonyloxy group which may be substituted with 1 to 3 hydroxyl groups or methoxy groups.

The flavan-3-ols represented by formula (2-1-A) are preferably those in which R⁴ and X are the same as defined above; R^{x1} is an aromatic hydrocarbon group which may be substituted with 1 to 3 hydroxyl groups or methoxy groups; R^{y1} is a hydrogen atom, a hydroxyl group, a methoxy group, or an arylcarbonyloxy or aralkylcarbonyloxy group which may be substituted with 1 to 3 hydroxyl groups or methoxy groups.

Examples of the compound of (2-1-A) include catechin, epicatechin, epigallocatechin, meciadanol, afzelechin, epiafzelechin, catechin gallate, epicatechin gallate, epigallocatechin gallate, phylloflavan, fisetinidol, guibourtinidol, and robinetinidol.

### (2-1-B) Flavan-4-ols Represented by the Following Formula (2-1-B)

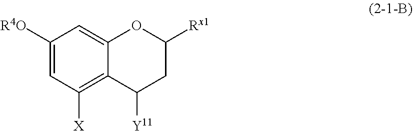

wherein R⁴, X, and R^{x1} are defined as above, and Y¹¹ represents a hydroxyl group or a methoxy group

The flavan-4-ol represented by formula (2-1-B) is preferably a compound wherein R⁴ and X are defined as above, Y¹¹ is a hydroxyl group, and R^{x1} is an aromatic hydrocarbon group which may be substituted with 1 to 3 hydroxyl groups or methoxy groups.

Examples of the compound of (2-1-B) include apiforol and luteoforol.

(2-1-C) Flavan-3,4-diols Represented by the Following Formula (2-1-C)

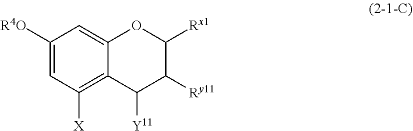

wherein R⁴, X, Y¹¹, R^{x1}, and R^{y11} are defined as above.

The flavan-3,4-diol represented by formula (2-1-C) is preferably a compound wherein R⁴ and X are defined as above; Y¹¹ is a hydroxyl group or a methoxy group; R^{x1} is an aromatic hydrocarbon group which may be substituted with 1 to 3 hydroxyl groups or methoxy groups; and R^{y1} is a hydroxyl group or a methoxy group.

Examples of the compound of (2-1-C) include leucocyanidin, leucodelphinidin, leucopelargonidin, leucopeonidin, and leucofisetinidin.

The compound of (2-2) is preferably any of the compounds represented by the following formula (2-2-A) or (2-2-B).

### (2-2-A) Flavanones Represented by the Following Formula (2-2-A)

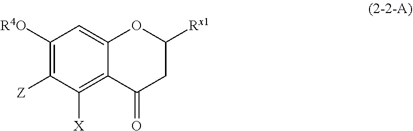

wherein R⁴, X, Z, and R^{x1} are defined as above, and

Z represents a C1 to C5 linear-chain or branched-chain alkyl or alkenyl group, only when R^{x1} is an o,p-dihydroxyaromatic hydrocarbon group, and represents a hydrogen atom in the other cases

The flavanone represented by formula (2-2-A) is preferably a compound wherein R⁴ and X are defined as above; Z is a hydrogen atom; and R^{x1} is an aromatic hydrocarbon group which may be substituted with 1 to 3 hydroxyl groups or methoxy groups; and R^{y1} is a hydroxyl group or a methoxy group.

Examples of the compound of (2-2-A) include eriodictyol, naringenin, pinocembrin, hesperetin, homoeriodictyol, isosakuranetin, sterubin, sakuranetin, alpinetin, and butin.

### (2-2-B) Flavanonols Represented by the Following Formula (2-2-B)

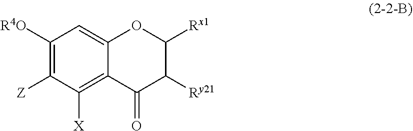

wherein R⁴, X, Z, and R^{x1} are defined as above; R^{y21} represents a hydroxyl group or a methoxy group;

Z represents a C1 to C5 linear-chain or branched-chain alkyl or alkenyl group, only when R^{x1} is an o,p-dihydroxyaromatic hydrocarbon group, and represents a hydrogen atom in the other cases

The flavanonol represented by formula (2-2-B) is preferably a compound wherein R⁴ and X are defined as above; Z is a hydrogen atom; R^{x1} is an aromatic hydrocarbon group which may be substituted with 1 to 3 hydroxyl groups or methoxy groups; and R^{y1} is a hydroxyl group or a methoxy group.

Examples of the compound of (2-2-B) include aromadendrin, taxifolin, and dihydrokaempferide.

The compound of (2-3) is preferably any of the compounds represented by the following formula (2-3-A) or (2-3-B).

### (2-3-A) Flavones Represented by the Following Formula (2-3-A)

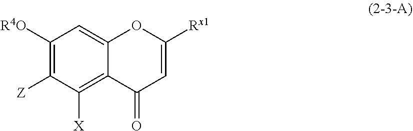

wherein R⁴, X, Z, and R^{x1} are defined as above; and

Z represents a C1 to C5 linear-chain or branched-chain alkyl or alkenyl group, only when R^{x1} is an o,p-dihydroxyaromatic hydrocarbon group, and represents a hydrogen atom in the other cases

The flavone represented by formula (2-3-A) is preferably a compound wherein R⁴ and X are defined as above; Z is a hydrogen atom; and R^{x1} is an aromatic hydrocarbon group which may be substituted with 1 to 3 hydroxyl groups or methoxy groups.

Examples of the compound of (2-3-A) include luteolin, apigenin, chrysin, norartocarpetin, tricetin, diosmetin, acacetin, chrysoeriol, genkwanin, techtochrysin, tricin, 4′,7-dihydroxyflavone, and pratol.

### (2-3-B) Flavonols Represented by Following Formula (2-3-B)

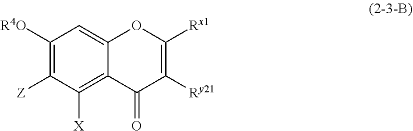

wherein R⁴, X, Z, R^{x1,} and R^{y21} are defined as above; and

Z represents a C1 to C5 linear-chain or branched-chain alkyl or alkenyl group, only when R^{x1} is an o,p-dihydroxyaromatic hydrocarbon group, and represents a hydrogen atom in the other cases

The flavonol represented by formula (2-3-B) is preferably a compound wherein R⁴ and X are defined as above; Z is a hydrogen atom; R^{x1} is an aromatic hydrocarbon group which may be substituted with 1 to 3 hydroxyl groups or methoxy groups; and R^{y1} is a hydroxyl group or a methoxy group.

Examples of the compound of (2-3-B) include quercetin, myricetin, morin, kaempferol, galangin, kaempferide, tamarixetin, laricitrin, annulatin, isorhamnetin, syringetin, rhamnetin, europetin, azaleatin, 5-O-methylmyricetin, retusin, pachypodol, rhamnazin, ayanin, ombuin, and fisetin.

The compound of (2-4) is preferably any of the compounds represented by the following formula (2-4-A) to (2-4-C).

### (2-4-A) Isoflavones Represented by the Following Formula (2-4-A)

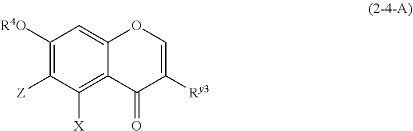

wherein R⁴, X, Z, and R^{y3} are defined as above; and

Z represents a C1 to C5 linear-chain or branched-chain alkyl or alkenyl group, only when R^{y3} is an o,p-dihydroxyaromatic hydrocarbon group, and represents a hydrogen atom in the other cases

The isoflavone represented by formula (2-4-A) is preferably a compound wherein R⁴ and X are defined as above; Z is a hydrogen atom or a C1 to C5 linear-chain or branched-chain alkyl or alkenyl group; and R^{y3} is an aromatic hydrocarbon group which may be substituted with 1 to 3 hydroxyl groups or methoxy groups and optionally forms a condensed ring with 1,3-dioxolane.

Examples of the compound of (2-4-A) include genistein, daidzein, orobol, biochanin A, pratensein, 5-O-methylgenistein, prunetin, calycosin, formononetin, 7-O-methylluteone, luteone, and pseudobaptigenin.

### (2-4-B) Isoflavans Represented by the Following Formula (2-4-B)

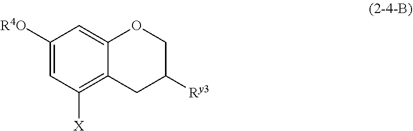

wherein R⁴, X, and R^{y3} are defined as above.

The isoflavan represented by formula (2-4-B) is preferably a compound wherein R⁴ and X are defined as above; and R^{y3} is an aromatic hydrocarbon group which may be substituted with 1 to 3 hydroxyl groups or methoxy groups and optionally forms a condensed ring with 1,3-dioxolane.

Examples of the compound of (2-4-B) include equol.

### (2-4-C) Isoflavenes Represented by the Following Formula (2-4-C)

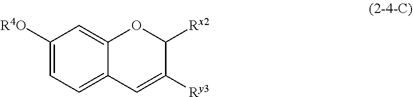

wherein R⁴, R^{x2}, and R^{y3} are defined as above.

The isoflavene represented by formula (2-4-C) is preferably a compound wherein R⁴ and R^{X2} are defined as above; and R^{y3} is an aromatic hydrocarbon group which may be substituted with 1 to 3 hydroxyl groups or methoxy groups and optionally forms a condensed ring with 1,3-dioxolane.

Examples of the compound of (2-4-C) include haginin D, haginin E, and 2-methoxyjudaicin.

Examples of the compound (2-5) include umbelliferone.

Among the aforementioned compounds represented by formula (2), preferred compounds are flavan-3-ols represented by formula (2-1-A), flavonols represented by formula (2-3-B), flavanones represented by formula (2-2-A), flavones represented by formula (2-3-A), isoflavones represented by formula (2-4-A), isoflavans represented by formula (2-4-B), and coumarins represented by formula (2-5). Specific examples of more preferred compounds include catechin, epicatechin, epigallocatechin, catechin gallate, epicatechin gallate, epigallocatechin gallate, quercetin, morin, hesperetin, naringenin, chrysin, daidzein, equol, and umbelliferone. Of these, catechin, epigallocatechin, epigallocatechin gallate, naringenin, and equol are yet more preferred. Mixtures such as a green tea, containing the aforementioned compounds, may also be used.

Each of the compounds represented by formula (2) preferably has a molecular weight of 150 or higher. From the viewpoint of permeability to hair, the molecular weight is preferably 1,000 or lower, more preferably 700 or lower, still more preferably 500 or lower.

Among the component (B), from the viewpoint of obtaining more remarkable change of the shape of hair after the treatment using the hair cosmetic composition of the present invention, improving shampooing resistance of shaped hair, obtaining more remarkable change of the shape of hair during semipermanent re-deforming of the shape of hair by heating, and improving shampooing resistance of shaped hair after re-deforming of the shape of hair by means of condensate of component (A) and component (B) formed in the hair, one or more member selected from the group consisting of resorcin derivatives represented by general formula (ii-1), m-dimethoxybenzene derivatives represented by general formula (1-1-1), resorcin derivatives represented by general formula (1-1-3), benzophenone derivatives represented by general formula (1-2), naphthol derivatives represented by general formula (1-3-a) or (1-3-b), flavan-3-ols represented by general formula (2-1-A), flavonols represented by general formula (2-3-B), flavanones represented by general formula (2-2-A), flavones represented by general formula (2-3-A), isoflavones represented by general formula (2-4-A), isoflavans represented by general formula (2-4-B) and coumarins represented by general formula (2-5) is preferable, and favorable compounds classified in each component is as described above.

Subcomponents of component (B) may be used singly or in combination of two or more species. Two or more of (B1) to (B3) may be used in combination. In the present invention, (B2) or (B3) is preferred, from the viewpoint of more consistently deforming hair.

In order to attain a remarkable change in hair shape after treatment with the hair cosmetic composition of the present invention, to further enhance the resistance of the hair shape to shampooing, to attain a more remarkable change in hair shape upon semi-permanently re-deforming of hair through heating, and to further enhance the resistance of the re-deformed hair to shampooing, the hair cosmetic composition of the present invention preferably has a total component (B) content, relative to the total hair cosmetic composition, of 0.2 mass % or higher, more preferably 0.5 mass % or higher, still more preferably 1.0 mass % or higher, yet more preferably 1.5 mass % or higher. In addition to the aforementioned viewpoints, in order to suitably mix components, the total component (B) content is preferably 40 mass % or lower, more preferably 30 mass % or lower, still more preferably 25 mass % or lower, yet more preferably 23 mass % or lower, further more preferably 20 mass % or lower.

In the case where component (B1) is used as component (B), the component (B1) content (i.e., the preferred component (B) content) of the hair cosmetic composition of the present invention, relative to the total hair cosmetic composition, is more preferably 2 mass % or higher, still more preferably 3 mass % or higher, yet more preferably 4 mass % or higher, further more preferably 5 mass % or higher, and in order to suitably mix components, more preferably 17 mass % or lower.

In the case where component (B2) is used as component (B), the component (B2) content (i.e., the preferred component (B) content) is more preferably 2 mass % or higher, and in order to suitably mix components, more preferably 17 mass % or lower, still more preferably 15 mass % or lower, yet more preferably 12 mass % or lower.

In the case where component (B3) is used as component (B), the component (B3) content (i.e., the preferred component (B) content) of the hair cosmetic composition of the present invention is more preferably 17 mass % or lower, still more preferably 15 mass % or lower, yet more preferably 12 mass % or lower, in order to suitably mix components.

In order to attain a remarkable change in hair shape after treatment with the hair cosmetic composition of the present invention by a condensation product between component (A) and component (B) formed in hair shafts, to further enhance the resistance of the hair shape to shampooing, to attain a more remarkable change in hair shape upon semi-permanently re-deforming of hair through heating, and to further enhance the resistance of the re-deformed hair to shampooing, the molar ratio of component (B) to component (A), (B)/(A), of the hair cosmetic composition of the present invention applied to hair is preferably 0.001 or higher, more preferably 0.1 or higher, still more preferably 0.2 or higher, yet more preferably 0.25 or higher, and preferably lower than 2.5, more preferably 2.3 or lower, still more preferably 2.1 or lower, yet more preferably 1.9 or lower, further more preferably 1.7 or lower, further more preferably 1.6 or lower.

In the case where component (B1) is used as component (B), the molar ratio of component (B1) to component (A), (B1)/(A), of the hair cosmetic composition of the present invention applied to hair is preferably 0.2 or higher, more preferably 0.3 or higher, still more preferably 0.4 or higher, yet more preferably 0.5 or higher, further more preferably 0.7 or higher, and preferably lower than 2.5, more preferably 2.3 or lower, still more preferably 2.0 or lower, yet more preferably 1.5 or lower, further more preferably 1.2 or lower.

In the case where component (B2) is used as component (B), the molar ratio of component (B2) to component (A), (B2)/(A), of the hair cosmetic composition of the present invention applied to hair is preferably 0.001 or higher, more preferably 0.1 or higher, still more preferably 0.2 or higher, yet more preferably 0.25 or higher, and preferably lower than 2.5, more preferably 2.3 or lower, still more preferably 2.0 or lower, yet more preferably 1.5 or lower, further more preferably 1.2 or lower.

In the case where component (B3) is used as component (B), the molar ratio of component (B3) to component (A), (B3)/(A), of the hair cosmetic composition of the present invention applied to hair is preferably 0.001 or higher, more preferably 0.1 or higher, still more preferably 0.2 or higher, yet more preferably 0.25 or higher, and preferably lower than 2.5, more preferably 2.3 or lower, still more preferably 2.0 or lower, yet more preferably 1.5 or lower.

### [Component (C): Direct Dye]

No particular limitation is imposed on the type of the direct dye of component (C), and any direct dye suitable for hair coloring may be used. Examples of the direct dye include an anionic dye, a nitro dye, a disperse dye, a cationic dye, an azophenol dye selected from the group consisting of the following HC Red 18, HC Blue 18, and HC Yellow 16, a salt thereof, and a mixture of any of these.

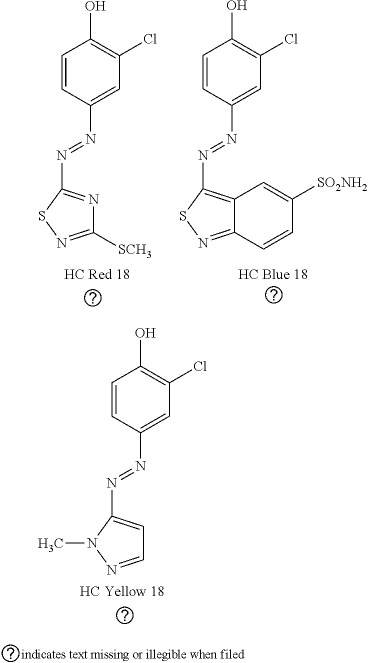 [SMILES: CSc1nsc(/N=N/c2ccc(O)c(Cl)c2)n1 ; NS(=O)(=O)c1ccc2nsc(/N=N/c3ccc(O)c(Cl)c3)c2c1 ; Cn1nccc1/N=N/c1ccc(O)c(Cl)c1]

Non-limitative examples of the cationic dye include Basic Blue 6, Basic Blue 7, Basic Blue 9, Basic Blue 26, Basic Blue 41, Basic Blue 99, Basic Brown 4, Basic Brown 16, Basic Brown 17, Natural Brown 7, Basic Green 1, Basic Orange 31, Basic Red 2, Basic Red 12, Basic Red 22, Basic Red 51, Basic Red 76, Basic Violet 1, Basic Violet 2, Basic Violet 3, Basic Violet 10, Basic Violet 14, Basic Yellow 57, Basic Yellow 87, and a mixture thereof. Of these, Basic Red 51, Basic Orange 31, Basic Yellow 87, and a mixture thereof are particularly preferred.

Non-limitative examples of the anionic dye include Acid Black 1, Acid Blue 1, Acid Blue 3, Food Blue 5, Acid Blue 7, Acid Blue 9, Acid Blue 74, Acid Orange 3, Acid Orange 4, Acid Orange 6, Acid Orange 7, Acid Orange 10, Acid Red 1, Acid Red 14, Acid Red 18, Acid Red 27, Acid Red 33, Acid Red 50, Acid Red 52, Acid Red 73, Acid Red 87, Acid Red 88, Acid Red 92, Acid Red 155, Acid Red 180, Acid Violet 2, Acid Violet 9, Acid Violet 43, Acid Violet 49, Acid Yellow 1, Acid Yellow 10, Acid Yellow 23, Acid Yellow 3, Food Yellow No. 8, D&C Brown No. 1, D&C Green No. 5, D&C Green No. 8, D&C Orange No. 4, D&C Orange No. 10, D&C Orange No. 11, D&C Red No. 21, D&C Red No. 27, D&C Red No. 33, D&C Violet No. 2, D&C Yellow No. 7, D&C Yellow No. 8, D&C Yellow No. 10, FD&C Red No. 2, FD&C Red No. 40, FD&C Red No. 4, FD&C Yellow No. 6, FD&C Blue No. 1, Food Black 1, Food Black 2, an alkali metal (e.g., sodium or potassium) salt, and a mixture of any of these.

Of these, preferred anionic dyes are Acid Black 1, Acid Red 52, Acid Violet 2, Acid Violet 43, Acid Red 33, Acid Orange 4, Acid Orange 7, Acid Red 27, Acid Yellow 3, Acid Yellow 10, and a salt thereof. More preferred anionic dyes are Acid Red 52, Acid Violet 2, Acid Red 33, Acid Orange 4, Acid Yellow 10, a salt thereof, and a mixture of any of these.

Non-limitative examples of the nitro dye include HC Blue No. 2, HC Blue No. 4, HC Blue No. 5, HC Blue No. 6, HC Blue No. 7, HC Blue No. 8, HC Blue No. 9, HC Blue No. 10, HC Blue No. 11, HC Blue No. 12, HC Blue No. 13, HC Brown No. 1, HC Brown No. 2, HC Green No. 1, HC Orange No. 1, HC Orange No. 2, HC Orange No. 3, HC Orange No. 5, HC Red BN, HC Red No. 1, HC Red No. 3, HC Red No. 7, HC Red No. 8, HC Red No. 9, HC Red No. 10, HC Red No. 11, HC Red No. 13, HC Red No. 54, HC Red No. 14, HC Violet BS, HC Violet No. 1, HC Violet No. 2, HC Yellow No. 2, HC Yellow No. 4, HC Yellow No. 5, HC Yellow No. 6, HC Yellow No. 7, HC Yellow No. 8, HC Yellow No. 9, HC Yellow No. 10, HC Yellow No. 11, HC Yellow No. 12, HC Yellow No. 13, HC Yellow No. 14, HC Yellow No. 15, 2-amino-6-chloro-4-nitrophenol, picramic acid, 1,2-diamino-4-nitrobenzole, 1,4-diamino-2-nitrobenzole, 3-nitro-4-aminophenol, 1-hydroxy-2-amino-3-nitrobenzole, 2-hydroxyethylpicramic acid, and a mixture thereof.

Non-limitative examples of the disperse dye include Disperse Blue 1, Disperse Black 9, Disperse Violet 1, and a mixture thereof.

Dyes of component (C) may be used singly or in combination of two or more species. Thus, the hair cosmetic composition of the present invention may contain therein a plurality of direct dyes having different ionic types. From the viewpoint of hair deforming performance, an anionic dye, a cationic dye, an azophenol dye selected from the group consisting of the following HC Red 18, HC Blue 18, and HC Yellow 16 are preferred, with an anionic dye and a cationic dye being more preferred.

From the viewpoint of attaining sufficient hair coloring performance, the component (C) content of the hair cosmetic composition of the present invention, relative to the total composition of the hair cosmetic composition, is preferably 0.001 mass % or higher, more preferably 0.01 mass % or higher, still more preferably 0.05 mass % or higher, and, from the viewpoint of suitably mixing components, preferably 10 mass % or lower, more preferably 7.5 mass % or lower, still more preferably 5 mass % or lower, yet more preferably 3 mass % or lower.

### [Component (D): Water]

The medium of the hair cosmetic composition of the present invention is (D) water. In the case of the multi-agent type composition, each of the first agent, the second agent, and the like employs (D) water as the medium.

The hair cosmetic composition of the present invention may be a one-agent type composition or a multi-agent type (e.g., a two-agent type) composition. However, in order to enhance permeability of components (A) and (B) to hair to attain enhanced effects of the present invention, a multi-agent type composition in which component (A) and component (B) are contained in different agents and which is used in the format of successive application. Further, a two-agent type composition is more preferred. In a preferred mode of the successive application-type multi-agent type composition, components (B) and (D) are incorporated into the first agent, which is firstly applied to the hair, and components (A) and (D) are incorporated into the second agent, which is applied to the hair after application of the first agent. In this mode, component (C) may be incorporated into the first agent or the second agent. However, component (C) is more preferably incorporated into the first agent, and is further more preferably incorporated into both the first and second agents.

[pH]

The pH of the hair cosmetic composition of the present invention, in the case of a one-agent type composition, is preferably 4.0 or lower, more preferably 3.0 or lower, still more preferably 2.5 or lower, yet more preferably 2.0 or lower, from the viewpoint of permeability to hair, and preferably 1.0 or higher, more preferably 1.2 or higher, still more preferably 1.5 or higher, from the viewpoints of suppression of damage to the hair and irritation to the skin. In the case of a multi-agent type composition, the pH of the agent containing component (A); i.e., the second agent, is preferably adjusted to fall within the aforementioned ranges. In the case of a multi-agent type composition, the pH of the agent containing component (B); i.e., the first agent, is preferably 6.0 or lower, more preferably 5.0 or lower, still more preferably 4.5 or lower, and preferably 2.5 or higher, more preferably 3.0 or higher, still more preferably 3.5 or higher, from the viewpoint of preventing discoloration of the composition. Notably, the pH of the hair cosmetic composition of the present invention refers to a pH value of a non-diluted or non-treated sample of the hair cosmetic composition measured at room temperature (25° C.) by means of a pH meter (model F-52, product of HORIBA).

In order to adjust the pH of the hair cosmetic composition to fall within the above ranges, a pH-adjusting agent may be appropriately used. Examples of the alkali pH-adjusting agent which may be used in the invention include ammonia and salts thereof; alkanolamines such as monoethanolamine, isopropanolamine, 2-amino-2-methylpropanol, and 2-aminobutanol, and salts thereof; alkane diamines such as 1,3-prpoanediamine, and salts thereof; carbonate salts such as guanidinium carbonate, sodium carbonate, potassium carbonate, sodium hydrogencarbonate, and potassium hydrogencarbonate; and hydroxides such as sodium hydroxide and potassium hydroxide. Examples of the acid pH-adjusting agent which may be used in the invention include inorganic acids such as hydrochloric acid and phosphoric acid; hydrochloric acid salts such as monoethanolamine hydrochloride; phosphate salts such as dihydrogen potassium phosphate and hydrogen disodium phosphate; organic acids other than component (A), such as lactic acid and malic acid.

### [Other Components]

In order to improve touch feeling to treated hair for further enhancing the effects of the present invention, one or more compositions forming the hair cosmetic composition preferably contain a cationic surfactant. The cationic surfactant is preferably a quaternary mono-long-chain alkylammonium salt having one C8 to C24 alkyl group and three C1 to C4 alkyl groups.

Preferably, at least one quaternary mono-long-chain alkylammonium surfactant is selected from the compounds represented by the following formula:

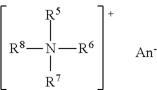

wherein R⁵ represents a C8 to C22 saturated or unsaturated linear-chain or branched-chain alkyl group, R⁹—CO—NH—(CH₂)ₘ—, or R⁹—CO—O—(CH₂)ₘ— (wherein R⁹ represents a C7 to C21 saturated or unsaturated linear-chain or branched-chain alky chain, and m is an integer of 1 to 4); R⁶, R⁷, and R⁸ each represent a C1 to C4 alkyl group or a C1 to C4 hydroxylalkyl group; and An⁻ represents a chloride ion, a bromide ion, a methosulfate ion, or an ethosulfate ion.

Examples of preferred cationic surfactants include long-chain quaternary ammonium compounds such as cetyltrimethylammonium chloride, myristyltrimethylammonium chloride, behenyltrimonium chloride, cetyltrimethylammonium bromide, and stearamidopropyltrimonium chloride. These compounds may be used singly or as a mixture thereof.

The cationic surfactant content of the hair cosmetic composition of the present invention, relative to the total composition of the hair cosmetic composition, is preferably 0.05 mass % or higher, more preferably 0.1 mass % or higher, and preferably 10 mass % or lower, more preferably 5 mass % or lower. In the case where the hair cosmetic composition is a multi-agent type composition, the cationic surfactant may be contained in the first agent and/or the second agent.

For improving touch feeling of treated hair and hair manageability, the hair cosmetic composition of the present invention preferably contains a silicone. The silicone is preferably dimethylpolysiloxane or an amino-modified silicone.

As the aforementioned dimethylpolysiloxane, any of cyclic and acyclic dimethylpolysiloxane polymers may be used. Examples of the dimethylpolysiloxane include SH200 series, BY22-019, BY22-020, BY11-026, B22-029, BY22-034, BY22-050A, BY22-055, BY22-060, BY22-083, and FZ-4188 (products of Dow Corning Toray), and KF-9088, KM-900 series, MK-15H, and MK-88 (products of Shin-Etsu Chemical Co., Ltd.).

As the aforementioned amino-modified silicone, any silicones having an amino group or an ammonium group may be used. Examples thereof include an amino-modified silicone oil in which the total or a part of terminal hydroxyl groups are end-capped with a methyl group or the like, and a non-end-capped amodimethicone. Among them, compounds represented by the following formula:

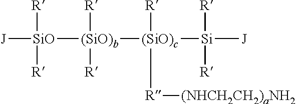

wherein R′ represents a hydrogen atom, a hydroxyl group, or R^{Z}; R^{Z} represents a substituted or non-substituted C1 to C20 monovalent hydrocarbon group; J represents R^{Z}, R″—(NHCH₂CH₂)ₐNH₂, OR^{Z}, or a hydroxyl group; R″ represents C1 to C8 divalent hydrocarbon group; a is a number of 0 to 3; and b+c (number average) is 10 or greater and less than 20,000, preferably 20 or greater and less than 3,000, more preferably 30 or greater and less than 1,000, still more preferably 40 or greater and less than 800] may be used as preferred amino-modified silicones.

Specific examples of preferred amino-modified silicone commercial products include amino-modified silicone oils such as SF8452C and SS3551 (products of Dow Corning Toray), KF-8004, KF-867S, and KF-8015 (products of Shin-Etsu Chemical Co., Ltd.), and amodimethicone emulsions such as SM8704C, SM8904, BY22-079, FZ-4671, and FZ-4672 (products of Dow Corning Toray).

The silicone content of the hair cosmetic composition of the present invention, relative to the total composition of the hair cosmetic composition, is preferably 0.1 mass % or higher, more preferably 0.2 mass % or higher, still more preferably 0.5 mass % or higher, and preferably 20 mass % or lower, more preferably 10 mass % or lower, still more preferably 5 mass % or lower. In the case where the hair cosmetic composition is a multi-agent type composition, the silicone may be contained in the first agent and/or the second agent.

In order to improve hair touch feeling after the hair treatment, one or more component compositions forming the hair cosmetic composition preferably contain a cationic polymer.

In the present invention, the “cationic polymer” refers to a polymer having a cationic group or a group which can be ionized to form a cationic group, and an amphoteric polymer having a totally positive charge. Examples of the cationic polymer include an aqueous polymer having an amino group or an ammonium group in polymer side chains and an aqueous polymer formed of structural repeating units of quaternary diallylammonium salt. Specific examples include cationized cellulose derivatives, cationic starch, cationic guar gum derivatives, quaternary diallylammonium salt polymers and copolymers, and quaternarized polyvinylpyrrolidone derivatives. Among them, from the viewpoints of attaining effects (mild touch feeling during rinsing and shampooing, smoothness and easy finger passage, high hair manageability during drying, and moisturizing effect) and stability of the treatment agents, polymers formed of structural repeating units of quaternary diallylammonium salt, quaternarized polyvinylpyrrolidone derivatives, and cationized cellulose derivatives are preferred, with quaternary diallylammonium salt polymers and copolymers and cationized cellulose derivatives being more preferred.

Specific examples of preferred quaternary diallylammonium salt polymers and copolymers include dimethyldiallylammonium chloride polymer (Polyquaternium-6) (e.g., Merquat 100; product of Lubrizol Advanced Materials), dimethyldiallylammonium chloride-acrylic acid copolymer (Polyquaternium-22) (e.g., Merquat 280 or 295; products of Lubrizol Advanced Materials), and dimethyldiallylammonium chloride-acrylamide copolymer (Polyquaternium-7) (e.g., Merquat 550; product of Lubrizol Advanced Materials).

Specific examples of preferred quaternarized polyvinylpyrrolidone derivatives include a polymer produced through polymerization of a vinylpyrrolidone copolymer with dimethylaminoethyl methacrylate (Polyquaternium-11) (e.g., Gafquat 734, Gafquat 755, or Gafquat 755N (products of Ashland).

Specific examples of preferred cationized cellulose derivatives include a polymer formed through addition of glycidyltrimethylammonium chloride to hydroxycellulose (Polyquaternium-10) (e.g., Leogard G or GP (products of Lion Specialty Chemicals Co., Ltd.), or Polymer JR-125, JR-400, JR-30M, LR-400, or LR-30M (products of Amerchol), and hydroxyethylcellulose dimethyldiallylammonium chloride (Polyquaternium-4) (e.g., Cell coat H-100 or L-200 (products of Akzo Nobel).

The cationic polymer content of the hair cosmetic composition of the present invention, relative to the total composition of the hair cosmetic composition, is preferably 0.001 mass % or higher, more preferably 0.01 mass % or higher, still more preferably 0.05 mass % or higher, and preferably 20 mass % or lower, more preferably 10 mass % or lower. In the case where the hair cosmetic composition is a multi-agent type hair cosmetic composition, the cationic polymer may be contained in the first agent and/or the second agent.

Optionally, an antioxidant may be added to one or more components of the hair cosmetic composition. The type and amount of the antioxidant may be those generally employed in the art. An example thereof is ascorbic acid.

In addition to the aforementioned components, the hair cosmetic composition of the present invention may further contain other appropriate components which are generally employed in hair cosmetic compositions. However, the hair cosmetic composition of the present invention preferably contains substantially no precursor, which is incorporated into an oxidation hair dye, the dye dyeing hair via oxidation reaction between the precursor and a coupler. In other words, the hair cosmetic composition of the present invention substantially contains no aromatic compound having at least one amino group in which one amino group has another amino group or a hydroxyl group at an o-position or the p-position of the aromatic ring relative to the first amino group, and having a closed-shell quinoid structure after oxidation. Any compound of component (B) has a structure similar to that of resorcin, which is a typical coupler of an oxidation hair dye. However, a technical feature of the present invention resides in that component (A) and component (B) are polymerized in hair shafts, whereby hair shape can be changed as desired through post heat treatment. Thus, the technical concept of the present invention completely differs from use of resorcin in an oxidation hair dye.

Yet preferably, the hair cosmetic composition of the present invention contains substantially no hair reducing agent. A characteristic feature of the present invention is to realize hair deforming not through breakage of S—S bonds of a protein in hair shafts. Therefore, the present invention absolutely differs from a permanent wave agent which breaks S—S bonds of hair shafts by the action of a reducing agent. Examples of the hair reducing agent include thiols such as thioglycolic acid, dithioglycolic acid, cysteine, acetylcysteine, and butyrolactone thiol, hydrogen sulfite, and a salt thereof.

In the present specification, the expression “substantially containing no specific compound” or an equivalent expression refers to the hair cosmetic composition having a target compound content, relative to the total composition of the hair cosmetic composition, of preferably less than 0.1 mass %, more preferably less than 0.01 mass %. Yet more preferably, the hair cosmetic composition contains no target compound.

The hair cosmetic composition of the present invention is highly safe to the human body and gives less damage to hair. Thus, the composition can be suitably applied particularly to human hair.

### [Hair Deforming Treatment Method]

The method for the treatment of hair, realizing semi-permanent or permanent deforming of hair, simultaneously dyeing hair, and employing the hair cosmetic composition of the present invention, may be carried out through a hair treatment method comprising the following steps (i) and (ii). Notably, the term “hair cosmetic composition” in the description in relation to the hair treatment refers to a composition actually applied to hair and encompasses any of a one-agent type hair cosmetic composition, a single application-type multi-agent type hair cosmetic composition which is a mixture of a first agent and a second agent, and a successive application-type multi-agent type hair cosmetic composition employing a first agent and a second agent.

The case of a one-agent type hair cosmetic composition:

(i) a step of applying a one-agent type hair cosmetic composition to hair; and

(ii) a step of heating and shaping the hair to which the hair cosmetic composition is applied.

The case of a multi-agent type hair cosmetic composition in a single application format:

(i) a step of mixing a first agent and a second agent of a multi-agent type hair cosmetic composition and applying the obtained hair cosmetic composition to hair; and

(ii) a step of heating and shaping the hair to which the hair cosmetic composition is applied.

The case of a multi-agent type hair cosmetic composition in a successive application format:

(i) a step of applying a first agent of a multi-agent type hair cosmetic composition to hair and then applying a second agent of the composition to a portion of the hair to which the first agent has been applied; and

(ii) a step of heating and shaping the hair to which the hair cosmetic composition is applied.

In step (i), the hair cosmetic composition may be applied to dry hair or wet hair. However, the target hair is preferably wetted with water before step (i) so as to swell the hair and promote permeation of the hair cosmetic composition to the hair. The relative mass of the hair cosmetic composition applied to hair in step (i); i.e., the mass ratio of liquid to hair (mass of hair cosmetic composition/mass of hair) is preferably 0.05 or higher, more preferably 0.10 or higher, still more preferably 0.25 or higher, yet more preferably 0.5 or higher, and preferably 5 or lower, more preferably 3 or lower, still more preferably 2 or lower. In the case of the successive application-type multi-agent type hair cosmetic composition, from the viewpoint of applicability, each of the first agent and the second agent preferably satisfies the above liquid-to-hair conditions. The target hair may be the total hair or a part of the hair.

In step (i), when the successive application-type multi-agent type hair cosmetic composition is used, the first agent is firstly applied. In order to promote permeation of the hair cosmetic composition and enhance the effects of the invention, the hair to which the first agent of the hair cosmetic composition has been applied may be allowed to stand for a certain period of time, and then the second agent may be further applied thereto. For suitably causing the hair cosmetic composition to permeate and diffuse in hair, the time of allowing to stand is preferably 1 minute or longer, more preferably 3 minutes or longer, still more preferably 5 minutes or longer, and preferably 1 hour or shorter, more preferably 30 minutes or shorter, still more preferably 20 minutes or shorter. In this case, in order to promote permeation of the first agent, the hair may be heated. The heating temperature is preferably 40 to 90° C.

In step (i), when the successive application-type multi-agent type hair cosmetic composition is used, a step of washing of the first agent (hereinafter may be referred to as an “intermediate rinsing step”) may be performed after application and staying of the first agent and before application of the second agent. From the viewpoint of shortening the total treatment time, the treatment method preferably includes no intermediate rinsing step. When no intermediate rinsing step is included, the molecular weight of component (B) contained in the first agent is preferably 100 to 180, more preferably 100 to 140, for further enhancing the effect for deforming the shape of hair. In contrast, from the viewpoint of enhancing touch feeling after hair deforming treatment, the treatment method preferably includes an intermediate rinsing step. When an intermediate rinsing step is included, the molecular weight of component (B) contained in the first agent is preferably 140 to 1,000, more preferably 180 to 1,000, for further enhancing the effect for deforming the shape of hair and attaining favorable touch feeling after hair deforming.

Also, when the successive application-type multi-agent type hair cosmetic composition is used, no particular limitation is imposed on the amounts of the first and second agents applied to hair. The molar ratio of component (B) to component (A) applied to hair, (B)/(A), is preferably 0.001 or higher, more preferably 0.1 or higher, still more preferably 0.2 or higher, and yet more preferably 0.25 or higher, and preferably lower than 2.5, more preferably 2.3 or lower, still more preferably 2.0 or lower, yet more preferably 1.5 or lower, in application of the hair cosmetic composition. In the present specification, each of the amounts of components (A) and (B) applied to hair is calculated from the amount of the first agent or the second agent applied to the hair, and the component (A) content or component (B) content of the first agent or the second agent.

An optional step of allowing the hair to which the hair cosmetic composition has been applied to stand for a specific period of time may be included between step (i) and step (ii). In this case, for suitably causing the hair cosmetic composition to permeate and diffuse in hair, the time of allowing to stand is preferably 1 minute or longer, more preferably 3 minutes or longer, still more preferably 5 minutes or longer, and preferably 1 hour or shorter, more preferably 30 minutes or shorter, still more preferably 20 minutes or shorter.

In the step of allowing the hair to stand, the hair may be heated for promoting permeation of the hair cosmetic composition. When heating is performed, the heating temperature is preferably 40 to 90° C. Though heating, a low-molecular-weight oligomer can be polymerized in hair shafts before step (ii). Thus, step (ii) can be performed more advantageously, which is preferred.

Between step (i) and step (ii), the hair to which the hair cosmetic composition has been applied may or may not be rinsed. However, no rinsing is preferably performed. In this case, the effects of sufficiently retaining components of the hair cosmetic composition in hair, imparting a semi-permanent shape to hair, and semi-permanently re-deforming the hair by heating can be further ensured.

For imparting a semi-permanent shape to hair and semi-permanently re-deforming the deformed hair by heat, the heating temperature in step (ii) is preferably 50° C. or higher, more preferably 60° C. or higher, still more preferably 80° C. or higher. In order to suppress rapid evaporation of water during heating, the heating temperature is preferably 250° C. or lower, more preferably 240° C. or lower, still more preferably 230° C. or lower. Examples of the heating means include a hair iron, an electrically heating rod, and a hot curler.

The heating time in step (ii) is appropriately adjusted in accordance with the apparatus and temperature employed in step (ii). However, in order to permeate and diffuse the hair cosmetic composition in hair shafts to induce satisfactory polymerization, the heating time is preferably 1 second or longer, more preferably 5 seconds or longer, still more preferably 1 minute or longer, yet more preferably 5 minutes or longer, further more preferably 15 minutes or longer, further more preferably 30 minutes or longer. From the viewpoint of suppression of damage to hair, the heating time is preferably 2 hours or shorter, more preferably 1 hour or shorter, still more preferably 45 minutes or shorter.

The shaping performed in step (ii) encompasses straightening and curling. Examples of the straightening method include blow-heating the hair by means of a hair drier, while drawing the hair by means of a tool such as the hand, a comb, or a brush, and heating the hair by means of a hair iron. From the viewpoint easiness of shaping, a hair iron is preferably employed. In one mode of straightening the hair by heating with a hair iron, the hair is nipped by a flat iron, and the flat iron is caused to slide from hair roots to hair ends. In another mode, the hair is nipped by a flat iron, while drawing the hair by means of a tool such as the hand, a comb, or a brush, and the hair is maintained in that state. Needless to say, the two modes may be combined. In curling the hair, examples of the curling method include heating the hair which is wound by electrically heating rods, a hot curler, or the like for a certain period of time, and winding the hair by a curl iron for a certain period of time.

Preferably, step (ii) is performed under the conditions where rapid water evaporation is suppressed. Specific means for suppressing evaporation of water include wrapping the hair to which the hair cosmetic composition has been applied with a plastic film (e.g., a food wrapping film), a cap, or the like, and continuously spraying steam (e.g., superheated steam) to the hair.

After carrying out step (ii), the shaped hair may or may not be rinsed. However, rinsing is preferably performed so as to prevent impairment hair touch feeling due to remaining polymers.

### (Hair Re-Deforming Method)

After the hair has been deformed through a method including step (i) or (ii), the deformed hair may be subjected to a step of semi-permanently deforming the hair to a different shape by heating (i.e., a re-deforming step). The heating temperature in re-deforming is preferably 30° C. or higher, more preferably 40° C. or higher, and preferably 230° C. or lower, more preferably 220° C. or lower, still more preferably 210° C. or lower. In the re-deforming of hair, application of the hair cosmetic composition of the present invention, a hair treatment agent containing a reducing agent (e.g., a permanent agent), or another type of hair treatment agent such as an alkali relaxer is preferably omitted.

Hereinafter, specific procedures of a step of semi-permanently deforming the hair to a different shape by heating will be described.

Re-Deforming of Shaped Curly Hair to Straight Hair

Examples of the method of re-deforming shaped curly hair to straight hair include blow-heating the hair by means of a hair drier, while drawing the hair by means of a tool such as the hand, a comb, or a brush, and heating the hair by means of a hair iron. From the viewpoint easiness of hair deforming, a hair iron is preferably employed. In one procedure of straightening the hair by heating with a hair iron, the hair is nipped by a hair iron, and the hair iron is caused to slide from hair roots to hair ends. In another procedure, the hair is nipped by a hair iron, while drawing the hair by means of a tool such as the hand, a comb, or a brush, and the hair is maintained in that state. The two procedures may be combined.

Under any conditions, including the type of the hair iron, the material of heating members thereof, the heating temperature employed, and the mode of operating the hair iron, in order to attain semi-permanent or permanent hair deforming, the actual temperature (hair temperature) during heating of the hair is preferably 120° C. or higher, more preferably 150° C. or higher. From the viewpoints of prevention of hair damage and deforming hair semi-permanently or permanently, the temperature is preferably 230° C. or lower, more preferably 220° C. or lower, still more preferably 210° C. or lower.

The temperature of heating hair can be monitored by means of, for example, a radiation thermometer (model ST653, product of SENTRY).

Re-Deforming of Straightened Hair to Curly Hair

Examples of the method of deforming straightened hair to curly hair include heating the hair which is wound by heating rods, a hot curler, or the like for a certain period of time, and winding the hair by a curl iron for a certain period of time.

In the re-deforming method, the actual temperature (hair temperature) during heating of the hair is preferably 30° C. or higher, more preferably 40° C. or higher, in order to suitably deforming the hair permanently or semi-permanently. From the viewpoints of prevention of hair damage and deforming hair semi-permanently or permanently, the temperature is preferably 180° C. or lower, more preferably 120° C. or lower, still more preferably 100° C. or lower, yet more preferably 80° C. or lower, further more preferably 60° C. or lower.

In re-deforming of hair, heating may be performed when the hair is dry or after the hair has been wetted with water. From the viewpoint of deforming hair semi-permanently or permanently, heating is preferably performed after the hair has been wetted with water.

The time of heating hair in hair re-deforming may be appropriately tuned in accordance with the heating tool, heating temperature, and other conditions employed in heating. From the viewpoint of deforming hair semi-permanently or permanently, the heating time is preferably 1 second or longer, more preferably 5 seconds or longer, still more preferably 1 minute or longer, yet more preferably 5 minutes or longer, further more preferably 15 minutes or longer, further more preferably 30 minutes or longer. In order to prevent hair damage, the heating time is preferably 2 hours or shorter, more preferably 1 hour or shorter, still more preferably 45 minutes or shorter.

The hair treatment method which can shape hair semi-permanently or permanently is not particularly limited to some embodiments. Preferred hair treatment methods include the following three embodiments (patterns).

Pattern 1: A case of one-agent type hair cosmetic composition

1) optionally wetting hair with water;

2) applying, to the hair, the hair cosmetic composition of the present invention containing the following components (A) to (D):

component (A): one or more compounds selected from the group consisting of glyoxylic acid, a glyoxylic acid hydrate, a glyoxylate salt, and a glyoxylamide;

component (B): a phenolic compound having an electron-donating group on at least one m-position and having a hydrogen atom on at least one of the o-positions and the p-position, wherein the electron-donating group on the m-position may form, together with an adjacent carbon atom, a benzene ring optionally substituted with hydroxyl group(s);

component (C): a direct dye; and

component (D): water;

3) optionally allowing the hair to which the hair cosmetic composition has been applied to stand for for 1 minute or more and 1 hour or less, wherein the hair may optionally be heated at 40 to 90° C.;

4) heating and shaping the hair at 50 to 250° C.;

5) optionally rinsing the hair;

6) optionally heating and reshaping the hair at 40 to 230° C.; and

7) optionally heating and further shaping the re-deformed hair at 40 to 230° C.

Pattern 2: A case of single application multi-agent type hair cosmetic composition

1) optionally wetting hair with water;

2) applying, to the hair, the hair cosmetic composition of the present invention containing the following components (A) to (D):

component (A): one or more compounds selected from the group consisting of glyoxylic acid, a glyoxylic acid hydrate, a glyoxylate salt, and a glyoxylamide;

component (B): a phenolic compound having an electron-donating group on at least one m-position and having a hydrogen atom on at least one of the o-positions and the p-position, wherein the electron-donating group on the m-position may form, together with an adjacent carbon atom, a benzene ring optionally substituted with hydroxyl group(s);

component (C): a direct dye; and

component (D): water, wherein the composition is prepared by mixing a first agent containing the components (B) and (D) and preferably additionally containing component (C), with a second agent preferably additionally containing component (C) (wherein the component (C) is essentially contained in at least one of the first and second agents);

3) optionally allowing the hair to which the hair cosmetic composition has been applied to stand for for 1 minute or more and 1 hour or less, wherein the hair may optionally be heated at 40 to 90° C.;

4) heating and shaping the hair at 50 to 250° C.;

5) optionally rinsing the hair;

6) optionally heating and reshaping the hair at 40 to 230° C.; and

7) optionally heating and further shaping the re-deformed hair at 40 to 230° C.

Pattern 3: A case of successive application multi-agent type hair cosmetic composition

1) optionally wetting hair with water;

2) applying, to hair, a first agent containing the following components (B) and (D), and preferably additionally containing component (C) (wherein the component (C) is essentially contained in at least one of the first and second agents):

component (B): a phenolic compound having an electron-donating group on at least one m-position and having a hydrogen atom on at least one of the o-positions and the p-position, wherein the electron-donating group on the m-position may form, together with an adjacent carbon atom, a benzene ring optionally substituted with hydroxyl group(s);

component (C): a direct dye; and

component (D): water;

3) optionally allowing the hair to which the hair cosmetic composition has been applied to stand for 1 minute or more and 1 hour or less, wherein the hair may optionally be heated at 40 to 90° C.;

4) optionally rinsing off the first agent remaining on the hair;

5) further applying, to a portion of the hair to which the first agent has been applied, a second agent containing the following components (A) and (D), and preferably additionally containing component (C):

component (A): one or more compounds selected from the group consisting of glyoxylic acid, a glyoxylic acid hydrate, a glyoxylate salt, and a glyoxylamide;

component (C): a direct dye; and

component (D): water;

6) optionally allowing the hair to which the hair cosmetic composition has been applied to stand for for 1 minute or more and 1 hour or less, wherein the hair may optionally be heated at 40 to 90° C.;

7) heating and shaping the hair at 50 to 250° C.;

8) optionally rinsing the hair;

9) optionally heating and reshaping the hair at 40 to 230° C.; and

10) optionally heating and further shaping the re-deformed hair at 40 to 230° C.

The technical feature of the hair treatment method of the present invention completely differs from conventional techniques such as a permanent treatment by use of a reducing agent and a relaxation treatment by use of a strong-alkali hair treatment agent having a pH of 12 to 14, and can shape hair to a form as desired. Thus, the hair treatment method of the present invention does not include a step of applying, to hair, a hair treatment agent containing a reducing agent or a strong-alkali hair treatment agent having a pH of 12 to 14. Therefore, as compared with conventional hair deforming methods, the hair treatment method according to the present invention is advantageous in that the hair can be deformed without damaging the hair.

In relation to the aforementioned embodiments, there will next be disclosed more preferred embodiments of the present invention.

<1>

A hair cosmetic composition, which is a one-agent type hair cosmetic composition composed of a single agent, or a multi-agent type hair cosmetic composition composed of multiple agents, wherein the hair cosmetic composition comprising the following components (A) to (D):

(A): one or more compounds selected from the group consisting of glyoxylic acid, a glyoxylic acid hydrate, a glyoxylate salt, and a glyoxylamide;

(B): a phenolic compound having an electron-donating group on at least one m-position, preferably two or three positions, and having a hydrogen atom on at least one of the o-positions and the p-position, wherein the electron-donating group on the m-position may form, together with an adjacent carbon atom, a benzene ring optionally substituted with hydroxyl group(s);

(C): a direct dye; and

(D): water.

<2>

The hair cosmetic composition as described in <1> above, wherein the molar ratio of component (B) to component (A), (B)/(A), of applied to hair is preferably 0.001 or higher, more preferably 0.1 or higher, still more preferably 0.2 or higher, yet more preferably 0.25 or higher, and preferably lower than 2.5, more preferably 2.3 or lower, still more preferably 2.1 or lower, yet more preferably 1.9 or lower, further more preferably 1.7 or lower, further more preferably 1.6 or lower.

<3>

The hair cosmetic composition as described in <1> or <2> above, wherein component (C) is preferably a species selected from the group consisting of an anionic dye, a nitro dye, a disperse dye, a cationic dye, an azophenol dye selected from the group consisting of HC Red 18, HC Blue 18, and HC Yellow 16, a salt thereof, or a compound of one or more of these species, more preferably, an anionic dye and a salt thereof.

<4>

The hair cosmetic composition as described in any one of <1> to <3> above, wherein the component (A) content, relative to the total composition of the hair cosmetic composition and in terms of glyoxylic acid, is preferably 1 mass % or higher, more preferably 2.0 mass % or higher, still more preferably 2.5 mass % or higher, yet more preferably 3.0 mass % or higher, and preferably 30 mass % or lower, more preferably 25 mass % or lower, still more preferably 20 mass % or lower, yet more preferably 15 mass % or lower, further more preferably 12 mass % or lower.

<5>

The hair cosmetic composition as described in any one of <1> to <4> above, wherein the component (B) content, relative to the total composition of the hair cosmetic composition, is preferably 0.2 mass % or higher, more preferably 0.5 mass % or higher, still more preferably 1.0 mass % or higher, yet more preferably 1.5 mass % or higher, and preferably 40 mass % or lower, more preferably 30 mass % or lower, still more preferably 25 mass % or lower, yet more preferably 23 mass % or lower, further more preferably 20 mass % or lower.

<6>

The hair cosmetic composition as described in any one of <1> to <5> above, wherein the component (C) content, relative to the total composition of the hair cosmetic composition, is preferably 0.001 mass % or higher, more preferably 0.01 mass % or higher, still more preferably 0.05 mass % or higher, and preferably 10 mass % or lower, more preferably 7.5 mass % or lower, still more preferably 5 mass % or lower.

<7>

The hair cosmetic composition as described in any one of <1> to <6> above, wherein component (B) is preferably resorcin (B1).

<8>

The hair cosmetic composition as described in <7> above, wherein the component (B1) content, relative to the total composition of the hair cosmetic composition, is preferably 0.2 mass % or higher, more preferably 0.5 mass % or higher, still more preferably 1.0 mass % or higher, yet more preferably 1.5 mass % or higher, further more preferably 2 mass % or higher, further more preferably 3 mass % or higher, further more preferably 4 mass % or higher, further more preferably 5 mass % or higher, and preferably 40 mass % or lower, more preferably 30 mass % or lower, still more preferably 25 mass % or lower, yet more preferably 20 mass % or lower, further more preferably 17 mass % or lower.

<9>

The hair cosmetic composition as described in <7> or <8> above, wherein the molar ratio of component (B1) to component (A), (B1)/(A), of the hair cosmetic composition applied to hair is preferably 0.2 or higher, more preferably 0.3 or higher, still more preferably 0.4 or higher, yet more preferably 0.5 or higher, further more preferably 0.7 or higher, and preferably lower than 2.5, more preferably 2.3 or lower, still more preferably 2.0 or lower, yet more preferably 1.5 or lower, further more preferably 1.2 or lower.

<10>

The hair cosmetic composition as described in any one of <1> to <6> above, wherein component (B) is preferably one or more compounds (B2) represented by the following formula (1):

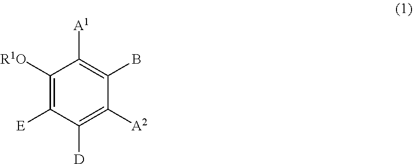

(wherein:

R¹ represents a hydrogen atom or a methyl group;

A¹ and A², which may be identical to or different from each other, each represent a hydrogen atom, a C1 to C12 linear-chain or branched-chain alkyl or alkenyl group, an optionally substituted C7 to C12 aralkyl or arylalkenyl group, a C1 to C6 linear-chain or branched-chain alkoxy or alkenyloxy group, a halogen atom, or —CO—R² (wherein R² represents a C1 to C12 linear-chain or branched-chain alkyl or alkenyl group, an optionally substituted C7 to C12 aralkyl or arylalkenyl group, or an optionally substituted C6 to C12 aromatic hydrocarbon group);

B represents a hydrogen atom, a C1 to C12 linear-chain or branched-chain alkyl or alkenyl group, an optionally substituted C7 to C12 aralkyl or arylalkenyl group, or —OR³ or —COOR³ (wherein R³ represents a hydrogen atom or a C1 to C6 linear-chain or branched-chain alkyl or alkenyl group);

D represents a hydrogen atom, a hydroxyl group, a methyl group, or a C1 to C12 linear-chain or branched-chain alkoxy or alkenyloxy group; and

E represents a hydrogen atom, a hydroxyl group, a C1 to C6 linear-chain or branched-chain alkyl or alkenyl group, or a C1 to C6 linear-chain or branched-chain alkoxy or alkenyloxy group,

wherein two or three of A¹, A², B, and E each are a hydrogen atom, with each of remaining being a group which is not a sulfo group, and in the case where D is a hydrogen atom or a methyl group, A¹ and B, or A² and B form an optionally hydroxyl group-substituted benzene ring with two adjacent carbon atoms), more preferably compounds represented by the following formula (1-1), (1-2), (1-3-a), or (1-3-b):

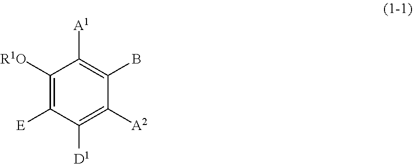

wherein R¹, A¹, A², B, and E are defined as above, and D¹ represents a hydroxyl group or a methoxy group;

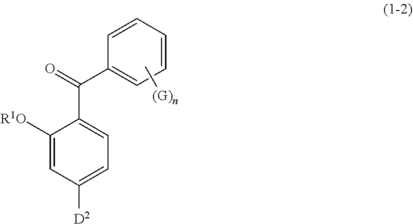

wherein R¹ has the same meaning as defined above; D² represents a hydroxyl group or a C1 to C12 alkoxy group; G represents a hydroxyl group, a C1 to C6 linear-chain or branched-chain alkyl or alkenyl group, or a C1 to C6 alkoxy group; and n is an integer of 0 to 2;

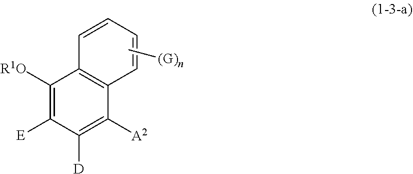

wherein R¹, A², E, D, G, and n are defined as above; or

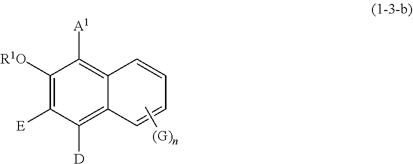

wherein R¹, A¹, E, D, G, and n are defined as above, more preferably one or more compounds represented by formula (1-1-1), (1-1-2), or (1-1-3):

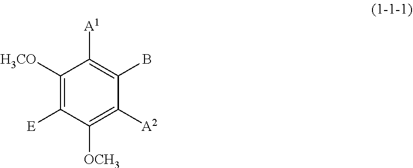

wherein A¹, A², B, and E are defined as above;

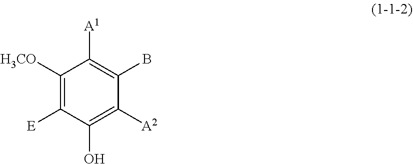

wherein A¹, A², B, and E are defined as above; or

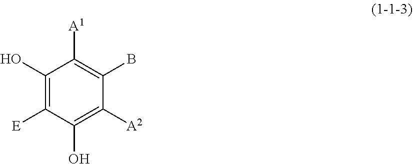

wherein A¹, A², B, and E are defined as above.
<11>

The hair cosmetic composition as described in <10> above, wherein component (B2) is preferably one or more species selected from the group consisting of 2-methylresorcin, 4-chlororesorcin, 4-alkylresorcin, 4-aralkylresorcin, 4-acylated resorcin, 5-alkylresorcin, 5-aralkylresorcin, 5-hydroxyarylalkenylresorcin, a 2,4,6-trihydroxyphenylaralkylketone, gallic acid, and a gallate ester; more preferably 4-butylresorcin (trivial name: rucinol), 4-(1-phenylethyl)resorcin (trivial name: Symwhite 377), 5-(hydroxyphenylethenyl)resorcin (trivial name: resveratrol), 3-hydroxyphenyl-1-(benzene-2,4,6-triol)propan-1-one (trivial name: phloretin), 4-(2,4-dihydroxybenzoyl)resorcin (trivial name: Benzophenone-2), 5-(hydroxyphenylethenyl)-1,3-dimethoxybenzene (trivial name: pterostilbene), and 1-naphthol; still more preferably 2-methylresorcin, 4-chlororesorcin, 1-naphthol, 4-n-butylresorcinol, 4-phenylresorcinol, 5-(hydroxyphenylethenyl)resorcin, 3-hydroxyphenyl-1-(benzene-2,4,6-triol)propan-1-one, and 4-(2,4-dihydroxybenzoyl) resorcin.

<12>

The hair cosmetic composition as described in <10> or <11> above, wherein the component (B2) content, relative to the total hair cosmetic composition, is preferably 0.2 mass % or higher, more preferably 0.5 mass % or higher, still more preferably 1.0 mass % or higher, yet more preferably 1.5 mass % or higher, further more preferably 2.0 mass % or higher, and from the viewpoint of easiness in mixing components, preferably 40 mass % or lower, more preferably 30 mass % or lower, still more preferably 25 mass % or lower, yet more preferably 23 mass % or lower, further more preferably 20 mass % or lower, further more preferably 17 mass % or lower, further more preferably 15 mass % or lower, further more preferably 12 mass % or lower.

<13>

The hair cosmetic composition as described in any one of <10> to <12> above, wherein the molar ratio of component (B2) to component (A), (B2)/(A), of the hair cosmetic composition applied to hair is preferably 0.001 or higher, more preferably 0.1 or higher, still more preferably 0.2 or higher, yet more preferably 0.25 or higher, and preferably lower than 2.5, more preferably 2.3 or lower, still more preferably 2.0 or lower, yet more preferably 1.5 or lower, further more preferably 1.2 or lower.

<14>

The hair cosmetic composition as described in any one of <1> to <6> above, wherein component (B) is preferably one or more compounds (B3) represented by the following formula (2):

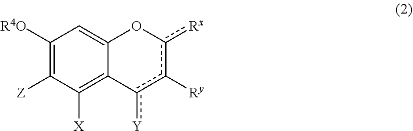

wherein,

R⁴ represents a hydrogen atom or a methyl group;

X represents a hydrogen atom, a hydroxyl group, or a methoxy group;

Y represents a hydrogen atom, an oxygen atom, a hydroxyl group, or a methoxy group;

Z represents a hydrogen atom or a C1 to C5 linear-chain or branched-chain alkyl or alkenyl group;

R^{x} represents a hydrogen atom, an oxygen atom, a hydroxyl group, a methoxy group, or an aromatic hydrocarbon group which may be substituted with 1 to 3 hydroxyl groups or methoxy groups and which optionally forms a condensed ring with 1,3-dioxolane;

R^{y} represents a hydrogen atom, a hydroxyl group, a methoxy group, an aromatic hydrocarbon group which may be substituted with 1 to 3 hydroxyl groups or methoxy groups and which optionally forms a condensed ring with 1,3-dioxolane, or an arylcarbonyloxy or aralkylcarbonyloxy group which may be substituted with 1 to 3 hydroxyl groups or methoxy groups;

a dotted line portion may be a double bond;

each of the combinations of the dotted line and the solid line being adjacent to R^{x} or Y denotes a double bond in the case where R^{x} or Y is an oxygen atom, and denotes a single bond in the other cases; and

Z represents a C1 to C5 linear-chain or branched-chain alkyl or alkenyl group, only when R^{x} or R^{y} is an o,p-dihydroxyaromatic hydrocarbon group, and represents a hydrogen atom in the other cases, more preferably one or more compounds represented by the following formula (2-1), (2-2), (2-3), (2-4), or (2-5);

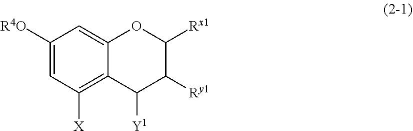

R⁴ and X are defined as above;

Y¹ represents a hydrogen atom, a hydroxyl group, or a methoxy group;

R^{x1} represents an aromatic hydrocarbon group which may be substituted with 1 to 3 hydroxyl groups or methoxy groups and which optionally forms a condensed ring with 1,3-dioxolane; and

R^{y1} represents a hydrogen atom, a hydroxyl group, a methoxy group, an aromatic hydrocarbon group which may be substituted with 1 to 3 hydroxyl groups or methoxy groups and which optionally forms a condensed ring with 1,3-dioxolane, or an arylcarbonyloxy or aralkylcarbonyloxy group which may be substituted with 1 to 3 hydroxyl groups or methoxy groups];

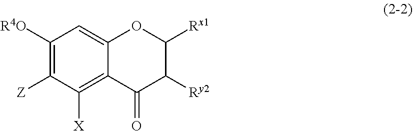

wherein R⁴, X, Z, and R^{x1} are defined as above, and R^{y2} represents a hydrogen atom, a hydroxyl group, or a methoxy group; and

Z represents a C1 to C5 linear-chain or branched-chain alkyl or alkenyl group, only when R^{x1} is an o,p-dihydroxyaromatic hydrocarbon group, and represents a hydrogen atom in the other cases;

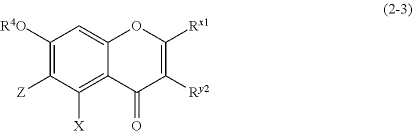

wherein R⁴, X, Z, R^{x1,} and R^{y2} are defined as above, and

Z is a C1 to C5 linear-chain or branched-chain alkyl or alkenyl group, only when R^{x1} is an o,p-dihydroxyaromatic hydrocarbon group. In the other cases, Z is a hydrogen atom;

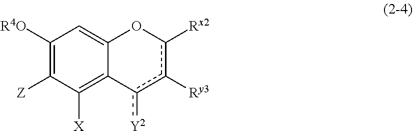

wherein

R⁴, X, Z, and the dotted line portion are defined as above;

Y² represents a hydrogen atom or an oxygen atom;

R^{x2} represents a hydrogen atom, a hydroxyl group, or a methoxy group;

R^{y3} represents an aromatic hydrocarbon group which may be substituted with 1 to 3 hydroxyl groups or methoxy groups and which optionally forms a condensed ring with 1,3-dioxolane;

the combination of the dotted line and the solid line being adjacent to Y² denotes a double bond in the case where Y² is an oxygen atom, and denotes a single bond in the other cases; and

Z represents a C1 to C5 linear-chain or branched-chain alkyl or alkenyl group, only when R^{y3} is an o,p-dihydroxyaromatic hydrocarbon group, and represents a hydrogen atom in the other cases; or

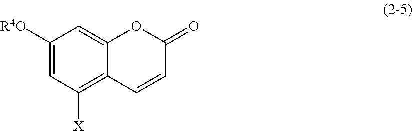

wherein R⁴ and X are defined as above.
<15>

The hair cosmetic composition as described in <14> above, wherein component (B3) is preferably one or more compounds selected from the group consisting of catechin, epicatechin, epigallocatechin, catechin gallate, epicatechin gallate, epigallocatechin gallate, quercetin, morin, hesperetin, naringenin, chrysin, daidzein, equol, and umbelliferone.

<16>

The hair cosmetic composition as described in <14> or <15> above, wherein the component (B3) content, relative to the total hair cosmetic composition, is preferably 0.2 mass % or higher, more preferably 0.5 mass % or higher, still more preferably 1.0 mass % or higher, yet more preferably 1.5 mass % or higher, and from the viewpoint of suitably mixing components, preferably 40 mass % or lower, more preferably 30 mass % or lower, still more preferably 25 mass % or lower, yet more preferably 23 mass % or lower, further more preferably 20 mass % or lower, further more preferably 17 mass % or lower further more preferably 15 mass % or lower, further more preferably 12 mass % or lower.

<17>

The hair cosmetic composition as described in any one of <14> to <16> above, wherein the molar ratio of component (B3) to component (A), (B3)/(A), of the hair cosmetic composition applied to hair is preferably 0.001 or higher, more preferably 0.1 or higher, still more preferably 0.2 or higher, yet more preferably 0.25 or higher, and preferably lower than 2.5, more preferably 2.3 or lower, still more preferably 2.0 or lower, yet more preferably 1.5 or lower.

<18>

The hair cosmetic composition as described in any one of <1> to <17> above, which is preferably a one-agent type hair cosmetic composition.

<19>

The hair cosmetic composition as described in <18> above, which has a pH of preferably 4.0 or lower, more preferably 3.0 or lower, still more preferably 2.5 or lower, yet more preferably 2.0 or lower, and preferably 1.0 or higher, more preferably 1.2 or higher, still more preferably 1.5 or higher.

<20>

The hair cosmetic composition as described in any one of <1> to <17> above, which is preferably a multi-agent type composition comprising a first agent containing components (B) and (D), and a second agent containing components (A) and (D), wherein component (C) is contained in the first agent and/or the second agent.

<21>

The hair cosmetic composition as described in <20> above, wherein the first agent preferably has a pH of 6.0 or lower, more preferably 5.0 or lower, still more preferably 4.5 or lower, and preferably 2.5 or higher, more preferably 3.0 or higher, still more preferably 3.5 or higher.

<22>

The hair cosmetic composition as described in <20> or <21> above, wherein the second agent preferably has a pH of 4.0 or lower, more preferably 3.0 or lower, still more preferably 2.5 or lower, yet more preferably 2.0 or lower, and preferably 1.0 or higher, more preferably 1.2 or higher, still more preferably 1.5 or higher.

<23>

The hair cosmetic composition as described in any one of <1> to <22> above, wherein one or more compositions forming the hair cosmetic composition preferably contain one or more species selected from the group consisting of a cationic surfactant, a silicone, and a cationic polymer.

<24>

A method for the treatment of hair comprising the following steps (i) and (ii);

(i) a step of applying the hair cosmetic composition as recited in <18> or <19> to hair; and

(ii) a step of heating and shaping the hair to which the hair cosmetic composition is applied.

<25>

The hair treatment method as described in <24>, the method comprising the following steps;

1) optionally wetting hair with water;

2) applying the hair cosmetic composition as recited in <18> or <19> to hair;

3) optionally allowing the hair to which the hair cosmetic composition has been applied to stand for 1 minute or more and 1 hour or less, wherein the hair may optionally be heated at 40° C. or higher and 90° C. or lower;

4) heating and shaping the hair at 50° C. or higher and 250° C. or lower;

5) optionally rinsing the hair;

6) optionally heating and reshaping the hair at 40° C. or higher and 230° C. or lower; and

7) optionally heating and further shaping the re-deformed hair at 40° C. or higher and 230° C. or lower.

<26>

A method for the treatment of hair comprising the following steps (i) and (ii);

(i) a step of mixing a first agent and a second agent of the hair cosmetic composition as recited in any one of <20> to <22> and applying the obtained hair cosmetic composition to hair; and

(ii) a step of heating and shaping the hair to which the hair cosmetic composition is applied.

<27>

The hair treatment method as described in <31>, the method comprising the following steps;

1) optionally wetting hair with water;

2) mixing the first agent and the second agent of the hair cosmetic composition as recited in any one of <20> to <22> and applying the obtained hair cosmetic composition to the hair;

3) optionally allowing the hair to which the hair cosmetic composition has been applied to stand for 1 minute or more and 1 hour or less, wherein the hair may optionally be heated at 40° C. or higher and 90° C. or lower;

4) heating and shaping the hair at 50° C. or higher and 250° C. or lower;

5) optionally rinsing the hair;

6) optionally heating and reshaping the hair at 40° C. or higher and 230° C. or lower; and

7) optionally heating and further shaping the re-deformed hair at 40° C. or higher and 230° C. or lower.

<28>

A method for the treatment of hair comprising the following steps (i) and (ii);

(i) a step of applying the first agent of the multi-agent type hair cosmetic composition as recited in any one of <20> to <22> to hair and then applying the second agent of the composition as recited in any one of <20> to <22> to a portion of the hair to which the first agent has been applied; and

(ii) a step of heating and shaping the hair to which the hair cosmetic composition is applied.

<29>

The hair treatment method as described in <33>, the method comprising the following steps;

1) optionally wetting hair with water;

2) applying, to the hair, the first agent of the multi-agent type hair cosmetic composition as recited in any one of <20> to <22>;

3) optionally allowing the hair to which the first agent has been applied to stand for 1 minute or more and 1 hour or less, wherein the hair may optionally be heated at 40° C. or higher and 90° C. or lower;

4) optionally rinsing the first agent applied onto the hair through rinsing the hair;

5) applying, to a portion of the hair to which the first agent has been applied, the second agent of the multi-agent type hair cosmetic composition as recited in any one of <20> to <22>;

6) optionally allowing the hair to which the second agent has been applied to stand for 1 minute or more and 1 hour or less, wherein the hair may optionally be heated at 40 or higher and 90° C. or lower;

7) heating and shaping the hair at 50° C. or higher and 250° C. or lower;

8) optionally rinsing the hair;

9) optionally heating and reshaping the hair at 40° C. or higher and 230° C. or lower; and

10) optionally heating and further shaping the re-deformed hair at 40° C. or higher and 230° C. or lower.

<30>

The hair treatment method as described in any one of <24> to <29>, wherein the step of heating and shaping the hair is preferably performed under the conditions where evaporation of water is suppressed.

<31>

The hair treatment method as described in any one of <24> to <30>, which method preferably includes no step of applying, to the hair, a hair treatment agent containing a reducing agent.

<32>

The hair treatment method as described in any one of <24> to <31>, which method preferably includes no step of applying, to the hair, a strongly alkaline hair treatment agent having a pH of 12 or higher and 14 or lower.

<33>

A hair cosmetic composition, which is a one-agent type hair cosmetic composition composed of a single agent, or a multi-agent type hair cosmetic composition composed of multiple agents,

wherein the hair cosmetic composition totally comprising the following components (A) to (D):

(A): 2.5 mass % or higher and 25 mass % or lower of one or more compound(s) selected from the group consisting of glyoxylic acid, glyoxylic acid hydrate, glyoxylate salt and glyoxylamide,

(B1): 5 mass % or higher and 40 mass % or lower of resorcin,

(C): 0.05 mass % or higher and 3.0 mass % or lower of one or more direct dye selected from the group consisting of anionic dye, cationic dye, nitro dye, HC Red 18, HC Blue 18, HC Yellow 16 and a salt thereof, and

(D): water;

wherein the molar ratio of component (B1) to component (A), (B1)/(A), is 0.5 or higher and 1.7 or lower,

wherein the pH of the hair cosmetic composition is 1.0 or higher and 3.0 or lower in the case where the hair cosmetic composition is the one-agent type composition, and wherein the pH of the first agent containing the component (B1) is 2.5 or higher and 5.0 or lower in the case where the hair cosmetic composition is the multi-agent type composition.

<34>

A hair treatment method for semi-permanently or permanently deforming hair shape, the method comprising the following steps (i) and (ii):

(i) a step of applying a hair cosmetic composition; and

(ii) a step of heating and shaping the hair to which the hair cosmetic composition is applied;

wherein the hair cosmetic composition is a one-agent type hair cosmetic composition composed of a single agent, or a multi-agent type hair cosmetic composition composed of multiple agents,

wherein the hair cosmetic composition totally comprising the following components (A) to (D):

(A): 2.5 mass % or higher and 25 mass % or lower of one or more compound(s) selected from the group consisting of glyoxylic acid, glyoxylic acid hydrate, glyoxylate salt and glyoxylamide,

(B1): 5 mass % or higher and 40 mass % or lower of resorcin,

(C): 0.05 mass % or higher and 3.0 mass % or lower of one or more direct dye selected from the group consisting of anionic dye, cationic dye, nitro dye, HC Red 18, HC Blue 18, HC Yellow 16 and a salt thereof, and

(D): water;

wherein the molar ratio of component (B1) to component (A), (B1)/(A), is 0.5 or higher and 1.7 or lower,

wherein the pH of the hair cosmetic composition is 1.0 or higher and 3.0 or lower in the case where the hair cosmetic composition is the one-agent type composition,

and wherein the pH of the first agent containing the component (B1) is 2.5 or higher and 5.0 or lower in the case where the hair cosmetic composition is the multi-agent type composition.

<35>

A hair cosmetic composition, which is a one-agent type hair cosmetic composition composed of a single agent, or a multi-agent type hair cosmetic composition composed of multiple agents,

wherein the hair cosmetic composition totally comprising the following components (A) to (D):

(A): 2.5 mass % or higher and 25 mass % or lower of one or more compound(s) selected from the group consisting of glyoxylic acid, glyoxylic acid hydrate, glyoxylate salt and glyoxylamide,

(B2): 5 mass % or higher and 30 mass % or lower of one or more phenolic compound(s) selected from the group consisting of 2-methylresorcin, 4-butylresorcin (trivial name: Rucinol), 4-hexylresorcin, 4-(1-phenylethyl)resorcin (trivial name: Symwhite377), 4-chlororesorcin, 5-(hydroxyphenylethenyl)resorcin (trivial name: resveratrol), 5-(hydroxyphenylethenyl)-1,3-dimethoxybenzene (trivial name: Pterostilbene), 3-hydroxyphenyl-1-(benzene-2,4,6-triol)propane-1-on (trivial name: Phloretin), 4-(2,4-dihydroxybenzoyl)resorcin (trivial name: Benzophenone-2) and 1-naphthol,

(C): 0.05 mass % or higher and 3.0 mass % or lower of one or more direct dye selected from the group consisting of anionic dye, cationic dye, nitro dye, HC Red 18, HC Blue 18, HC Yellow 16 and a salt thereof, and

(D): water;

wherein the molar ratio of component (B2) to component (A), (B2)/(A), is 0.1 or higher and 2.0 or lower,

wherein the pH of the hair cosmetic composition is 1.0 or higher and 3.0 or lower in the case where the hair cosmetic composition is the one-agent type composition,

and wherein the pH of the first agent containing the component (B2) is 2.5 or higher and 5.0 or lower in the case where the hair cosmetic composition is the multi-agent type composition.

<36>

A hair treatment method for semi-permanently or permanently deforming hair shape, the method comprising the following steps (i) and (ii):

(i) a step of applying a hair cosmetic composition; and

(ii) a step of heating and shaping the hair to which the hair cosmetic composition is applied;

wherein the hair cosmetic composition is a one-agent type hair cosmetic composition composed of a single agent, or a multi-agent type hair cosmetic composition composed of multiple agents,

wherein the hair cosmetic composition totally comprising the following components (A) to (D):

(A): 2.5 mass % or higher and 25 mass % or lower of one or more compound(s) selected from the group consisting of glyoxylic acid, glyoxylic acid hydrate, glyoxylate salt and glyoxylamide,

(B2): 5 mass % or higher and 30 mass % or lower of one or more phenolic compound(s) selected from the group consisting of 2-methylresorcin, 4-butylresorcin (trivial name: Rucinol), 4-hexylresorcin, 4-(1-phenylethyl)resorcin (trivial name: Symwhite377), 4-chlororesorcin, 5-(hydroxyphenylethenyl)resorcin (trivial name: resveratrol), 5-(hydroxyphenylethenyl)-1,3-dimethoxybenzene (trivial name: Pterostilbene), 3-hydroxyphenyl-1-(benzene-2,4,6-triol)propane-1-on (trivial name: Phloretin), 4-(2,4-dihydroxybenzoyl)resorcin (trivial name: Benzophenone-2) and 1-naphthol,

(C): 0.05 mass % or higher and 3.0 mass % or lower of one or more direct dye selected from the group consisting of anionic dye, cationic dye, nitro dye, HC Red 18, HC Blue 18, HC Yellow 16 and a salt thereof, and

(D): water;

wherein the molar ratio of component (B2) to component (A), (B2)/(A), is 0.1 or higher and 2.0 or lower,

wherein the pH of the hair cosmetic composition is 1.0 or higher and 3.0 or lower in the case where the hair cosmetic composition is the one-agent type composition,

and wherein the pH of the first agent containing the component (B2) is 2.5 or higher and 5.0 or lower in the case where the hair cosmetic composition is the multi-agent type composition.

<37>

A hair cosmetic composition, which is a one-agent type hair cosmetic composition composed of a single agent, or a multi-agent type hair cosmetic composition composed of multiple agents,

wherein the hair cosmetic composition totally comprising the following components (A) to (D):

(A): 2.5 mass % or higher and 25 mass % or lower of one or more compound(s) selected from the group consisting of glyoxylic acid, glyoxylic acid hydrate, glyoxylate salt and glyoxylamide,

(B3): 5 mass % or higher and 30 mass % or lower of one or more phenolic compound(s) selected from the group consisting of catechin, epigallocatechin, epigallocatechin gallate, naringenin and equol,

(C): 0.05 mass % or higher and 3.0 mass % or lower of one or more direct dye selected from the group consisting of anionic dye, cationic dye, nitro dye, HC Red 18, HC Blue 18, HC Yellow 16 and a salt thereof, and

(D): water;

wherein the molar ratio of component (B2) to component (A), (B3)/(A), is 0.1 or higher and 2.0 or lower,

wherein the pH of the hair cosmetic composition is 1.0 or higher and 3.0 or lower in the case where the hair cosmetic composition is the one-agent type composition,

and wherein the pH of the first agent containing the component (B3) is 2.5 or higher and 5.0 or lower in the case where the hair cosmetic composition is the multi-agent type composition.

<38>

A hair treatment method for semi-permanently or permanently deforming hair shape, the method comprising the following steps (i) and (ii):

(i) a step of applying a hair cosmetic composition; and

(ii) a step of heating and shaping the hair to which the hair cosmetic composition is applied;

wherein the hair cosmetic composition is a one-agent type hair cosmetic composition composed of a single agent, or a multi-agent type hair cosmetic composition composed of multiple agents,

wherein the hair cosmetic composition totally comprising the following components (A) to (D):

(A): 2.5 mass % or higher and 25 mass % or lower of one or more compound(s) selected from the group consisting of glyoxylic acid, glyoxylic acid hydrate, glyoxylate salt and glyoxylamide,

(B3): 5 mass % or higher and 30 mass % or lower of one or more phenolic compound(s) selected from the group consisting of catechin, epigallocatechin, epigallocatechin gallate, naringenin and equol,

(C): 0.05 mass % or higher and 3.0 mass % or lower of one or more direct dye selected from the group consisting of anionic dye, cationic dye, nitro dye, HC Red 18, HC Blue 18, HC Yellow 16 and a salt thereof, and

(D): water;

wherein the molar ratio of component (B2) to component (A), (B3)/(A), is 0.1 or higher and 2.0 or lower,

wherein the pH of the hair cosmetic composition is 1.0 or higher and 3.0 or lower in the case where the hair cosmetic composition is the one-agent type composition,

and wherein the pH of the first agent containing the component (B3) is 2.5 or higher and 5.0 or lower in the case where the hair cosmetic composition is the multi-agent type composition.

<39>

The hair cosmetic composition as described in <33>, <35> or <37>, wherein the amount of precursor is preferably 0.1 mass % or lower, more preferably the composition contains substantially no precursor.

<40>

The hair treatment method as described in <34>, <36> or <38>, wherein the amount of precursor in the hair cosmetic composition is preferably 0.1 mass % or lower, more preferably the composition contains substantially no precursor.

<41>

The hair cosmetic composition as described in <33>, <35> or <37>, wherein the amount of boric acid and silicic acid is preferably 0.1 mass % or lower, more preferably the composition contains substantially no boric acid and silicic acid.

<42>

The hair treatment method as described in <34>, <36> or <38>, wherein the amount of boric acid and silicic acid in the hair cosmetic composition is preferably 0.1 mass % or lower, more preferably the composition contains substantially no boric acid and silicic acid.

<43>

The hair cosmetic composition as described in <33>, <35> or <37>, wherein the amount of hair reducing agent is preferably 0.1 mass % or lower, more preferably the composition contains substantially no hair reducing agent.

<44>

The hair treatment method as described in <34>, <36> or <38>, wherein the amount of hair reducing agent in the hair cosmetic composition is preferably 0.1 mass % or lower, more preferably the composition contains substantially no hair reducing agent.

<45>

Use of a hair cosmetic composition, which is a one-agent type hair cosmetic composition composed of a single agent, or a multi-agent type hair cosmetic composition composed of multiple agent, and which totally comprises the following components (A) to (D):

(A): one or more compounds selected from the group consisting of glyoxylic acid, a glyoxylic acid hydrate, a glyoxylate salt, and a glyoxylamide;

(B): a phenolic compound having an electron-donating group on at least one m-position, preferably two or three positions, and having a hydrogen atom on at least one of the o-positions and the p-position, wherein the electron-donating group on the m-position may form, together with an adjacent carbon atom, a benzene ring optionally substituted with hydroxyl group(s);

(C): a direct dye; and

(D): water,

for semi-permanently or permanently deforming the shape of hair.

### EXAMPLES

### Example 1 and Comparative Example 1

One-agent type hair cosmetic compositions shown in Table 1 were prepared. Shape-giving effect and dyeing performance were evaluated through the following procedures and on the basis of the following criteria. The results are also shown in Table 1. The pH of each composition was obtained from a sample of the composition which was not diluted after preparation and was measured at room temperature (25° C.) by means of a pH meter (model F-52, product of HORIBA).

### <I: Semi-Permanent Curling (Initial Shape-Giving Effect)>

1. A 25-cm tress of Caucasian straight hair (untreated) (0.5 g) was wetted with tap water at 30° C. for 30 seconds, and the wetted hair tress was wound around a plastic rod (diameter: 14 mm), followed by fixing with a clip.

2. Onto the thus-wound hair tress, each hair cosmetic composition (1 g) was applied, and the hair tress wound over the rod was tightly wrapped by a plastic film. The sample was heated in an oven at 90° C. for 1 hour.

3. The tress was removed from the oven, and cooled to a room temperature.

4. The tress was removed from the rod, and was then rinsed with running tap water at 30° C. for 30 seconds. Thereafter, a shampoo for assessment was lathered on the tress for 60 seconds.

5. The tress was rinsed with running tap water at 30° C. for 30 seconds, and immersed at an infinite bath ratio in tap water at 30° C. for 60 seconds. Thereafter, the tress was gently pulled up out of the water while holding the root thereof, and water was then drained off by lightly shaking it.

6. The hair tress sample was allowed to stand in a laboratory for 2 hours, while the sample was suspended, and dried, followed by combing. Thereafter, the length of the hair tress sample perpendicularly suspended (i.e., the distance from the root of the sample to a point most distant therefrom) was measured.

The percent decrease in tress length (I, %), obtained from the length of the untreated tress sample (L₀) and the length of the treated tress sample (L) by the following equation was defined as the curling strength. The sample was evaluated on the basis of the curling strength. The sample was classified into five grades A through E on the basis of the following criteria.

I=[(L₀−L)/L₀]×100

### A: 20≤I

### B: 7.5≤I<20

### C: 5≤I<7.5

### D: 2.5≤I<5

### E: I<2.5

### <Dyeing Performance>

1. A 10-cm tress of white hair (untreated) (1.0 g) was wetted with tap water at 30° C. for 30 seconds, and the water was lightly removed by a towel.

2. The tress was laid on a plastic wrap and 1.5 g of the treatment agent was applied thereto and distributed evenly, and another wrap was covered over the tress, fixed by mending tape to hermetically seal the tress in the cavity formed, and heated at 90° C. for 1 hour in an oven.

3. The hermetically sealed tress in the wrap was removed from the oven, and cooled to a room temperature.

4. The tress was removed from the wrap, and was then rinsed with running tap water at 30° C. for 30 seconds. Thereafter, a shampoo for assessment was lathered on the tress for 60 seconds. The tress was rinsed with running tap water at 30° C. for 30 seconds, and dried for 5 minutes using cold blowing by drier while combing.

5. The colors of the front and back sides of the tress were measured at the vicinity of the root, intermediate, and end portion of the tress through use of a colorimeter (colorimeter CR-400, product of Konica Minolta, Inc.). The average of colorimetric values at 6 points in total was employed as a measured colorimetric value (L, a, b).

6. The degree of dyeing was evaluated by ΔE*ab, with Comparative Example 1 which does not contain the component (C) used as a reference. The color measurement was performed on the day on which the tress was dyed.

(Formulation of shampoo for assessment)

| Components | (mass %) |
|---|---|
| Sodium laureth sulfate | 15.5 |
| Lauramide DEA | 1.5 |
| Sodium benzoate | 0.5 |
| EDTA-2Na | 0.3 |
| Phosphoric acid | amount to pH 7 |
| Deionized water | balance |
| Total | 100 |

TABLE 1

| | | | | Comparative |
|---|---|---|---|---|
| | | | Example | Example |
| (mass %) | | | 1 | 1 |
| Formulation | (B) | Resorcin | 30 | 30 |
| [mass %] | (A) | Glyoxylic acid | 20 | 20 |
| | (C) | Acid Red 33 | 0.5 | — |
| | — | Sodium hydroxide | * | * |
| | (D) | Purified water | bal | bal |

| | Total | 100 | 100 |
|---|---|---|---|
| pH | | 2.0 | 2.0 |
| Molar ratio (B)/(A) | | 1.0 | 1.0 |
| Shape-giving | Curl tightness | 10.3 | 8.4 |
| | (decrease in tress length (%)) | | |
| | Shape-giving effect | B | B |
| Dyeing | ΔE*ab | 73.2 | standard |
| performance | | | |

* Amount for adjusting pH

### Examples 2 to 6 and Comparative Examples 2 to 4

Two-agent type hair cosmetic compositions (Examples 2 to 6 and Comparative Examples 2 to 3) shown in Table 2 were prepared. Shape-giving effect, dyeing performance, and color fastness were evaluated through the following procedures and on the basis of the following criteria. Notably, of the above-mentioned two-agent type hair cosmetic compositions, only Example 6 and Comparative Example 3 were evaluated for color fastness. Further, assessment was also made for the case where treatment was performed through sole use of the first agent which did not contain the component (A) (Comparative Example 4). The results are also shown in Table 2.

### <I: Semi-Permanent Curling (Initial Shape-Giving Effect)>

1. A 25-cm tress of Caucasian straight hair (untreated) (0.5 g) was wetted with tap water at 30° C. for 30 seconds, and the wetted hair tress was wound around a plastic rod (diameter: 14 mm), followed by fixing with a clip.

2. Onto the thus-wound hair tress, a first agent formulation (1 g) was applied, and the hair tress wound over the rod was tightly wrapped by a plastic film. The sample was heated in an oven at 90° C. for 1 hour.

3. The hair tress sample was removed from the oven and cooled to room temperature.

4. The tress was removed from the rod, and was then rinsed with running tap water at 30° C. for 30 seconds. Thereafter, a shampoo for assessment was lathered on the tress for 60 seconds, and was then rinsed with running tap water at 30° C. for 30 seconds.

5. The wet hair tress was wound around a plastic rod (diameter: 14 mm) and fixed with a clip.

6. Onto the thus-wound hair tress, a second agent formulation (1 g) was applied, and the hair tress wound over the rod was tightly wrapped by a plastic film. The sample was heated in an oven at 90° C. for 1 hour.

7. The hair tress sample was removed from the oven and cooled to room temperature.

8. The hair tress sample was released from the rod and rinsed with a flow of tap water at 30° C. for 30 seconds. Then, a shampoo for assessment was lathered on the tress for 60 seconds.

9. The tress was rinsed with running tap water at 30° C. for 30 seconds, and immersed at an infinite bath ratio in tap water at 30° C. for 60 seconds. Thereafter, the tress was gently pulled up out of the water while holding the root thereof, and water was then drained off by lightly shaking it.

10. The hair tress sample was allowed to stand in a laboratory for 2 hours, while the sample was suspended, and dried, followed by combing. Thereafter, the length of the hair tress sample perpendicularly suspended (i.e., the distance from the root of the sample to a point most distant therefrom) was measured.

The percent decrease in tress length (I, %), obtained from the length of the untreated tress sample (L₀) and the length of the treated tress sample (L) by the following equation was defined as the curling strength. The sample was evaluated on the basis of the curling strength. The sample was classified into five grades A through E on the basis of the following criteria.

I=[(L₀−L)/L₀]×100

### A: 20≤I

### B: 7.5≤I<20

### C: 5≤I<7.5

### D: 2.5≤I<5

### E: I<2.5

### <Dyeing Performance>

1. A 10-cm tress of white hair (untreated) (1.0 g) was wetted with tap water at 30° C. for 30 seconds, and the water was lightly removed by a towel.

2. The tress was laid on a plastic wrap and 1.5 g of the first agent was applied thereto and distributed evenly, and another wrap was covered over the tress, fixed by mending tape to hermetically seal the tress in the cavity formed, and heated at 90° C. for 1 hour in an oven.

3. The hermetically sealed tress in the wrap was removed from the oven, and cooled to a room temperature.

4. The tress was removed from the wrap, and was then rinsed with running tap water at 30° C. for 30 seconds. Thereafter, a shampoo for assessment was lathered on the tress for 60 seconds. The tress was rinsed with running tap water at 30° C. for 30 seconds, and dried for 5 minutes using cold blowing by drier while combing.

5. The tress was laid on a plastic wrap and 1.5 g of the second agent was applied thereto and distributed evenly, and another wrap was covered over the tress, fixed by mending tape to hermetically seal the tress in the cavity formed, and heated at 90° C. for 1 hour in an oven.

6. The tress sealed by the pieces of plastic wrap was removed from the oven and cooled to room temperature.

7. The tress was removed from the wrap, and was then rinsed with running tap water at 30° C. for 30 seconds. Thereafter, a shampoo for assessment was lathered on the tress for 60 seconds. The tress was rinsed with running tap water at 30° C. for 30 seconds, and dried for 5 minutes using cold blowing by drier while combing.

8. The colors of the front and back sides of the tress were measured at the vicinity of the root, intermediate, and end portion of the tress through use of a colorimeter (colorimeter CR-400, product of Konica Minolta, Inc.). The average of colorimetric values at 6 points in total was employed as a measured colorimetric value (L, a, b).

9. The degree of dyeing was evaluated by ΔE*ab, with Comparative Example 1 which does not contain the component (C) used as a reference. The color measurement was performed on the day on which the tress was dyed.

Notably, the aforementioned shampoo for assessment was used.

TABLE 2

| | | | | Examples | | | | | Comparative Examples | | |
|---|---|---|---|---|---|---|---|---|---|---|---|
| (mass %) | | | | 2 | 3 | 4 | 5 | 6 | 2 | 3 | 4 |
| First | Formulation | (B) | 4-Phenylethylresorcin | 10 | — | — | — | — | 10 | — | — |
| agent | [mass %] | | (+)-Catechin | — | 10 | 10 | 10 | 10 | — | 10 | — |
| | | (C) | Acid Red 33 | 0.5 | — | — | — | 0.5 | — | — | 0.5 |
| | | | Basic Red 51 | — | 0.5 | — | — | — | — | — | — |
| | | | HC Red BN | — | — | 0.5 | — | — | — | — | — |
| | | | HC Red 18 | — | — | — | 0.01 | — | — | — | — |
| | | — | Ethanol | 40 | 40 | 40 | 80 | 20 | 40 | 20 | 20 |
| | | | Hydrochloric acid or | * | * | * | * | * | * | * | * |
| | | | Sodium hydroxide | | | | | | | | |
| | | (D) | Purified Water | bal | bal | bal | bal | bal | bal | bal | bal |
| | | Total | | 100 | 100 | 100 | 100 | 100 | 100 | 100 | 100 |
| | pH | | | 4.0 | 4.0 | 4.0 | 4.0 | 4.0 | 4.0 | 4.0 | 4.0 |
| Second agent | Formulation | (A) | Glyoxylic acid | 10 | 10 | 10 | 10 | 10 | 10 | 10 | — |
| | [mass %] | — | Sodium hydroxide | * | * | * | * | * | * | * | |
| | | (D) | Purified Water | bal | bal | bal | bal | bal | bal | bal | |

| | | | | | | | |
|---|---|---|---|---|---|---|---|
| Total | 100 | 100 | 100 | 100 | 100 | 100 | 100 |

| | pH | | 2.0 | 2.0 | 2.0 | 2.0 | 2.0 | 2.0 | 2.0 | |
|---|---|---|---|---|---|---|---|---|---|---|
| Molar ratio (B)/(A) | | | 0.34 | 0.25 | 0.25 | 0.25 | 0.25 | 0.34 | 0.25 | — |
| Assessment | Shape-giving | Curl tightness | 16.4 | 14.1 | 11.4 | 16.4 | 13.2 | 14.1 | 13.2 | — |
| | | (decrease in tress length | | | | | | | | |
| | | (%)) | | | | | | | | |
| | | Shape-giving effect | B | B | B | B | B | B | B | — |
| | Dyeing performance | ΔE*ab | 66.5 | 41.2 | 18.1 | 5.8 | 50.6 | standard | standard | — |

*: Amount for adjusting pH

### <Fastness Test>

For Example 6 and Comparative Examples 3 and 4 of Table 2, the fastness of dyeing was tested in accordance with the following procedure.

1. 40 g of deionized water was poured into a plastic bottle (Sampler® PP wide-mouthed bottle (50 ml), the tress of the white hair used for the dyeing assessment was immersed into the water, and a cap was attached to the bottle.

2. The bottle including the water and the tress was placed in an oven of 40° C. for heating for 84 hours.

3. The bottle was removed from the oven and cooled to a room temperature.

4. The cap was removed from the bottle, and the white hair tress was removed. The amount of the dye leaked from the tress was evaluated on the basis of the degree of dyeing of the water in the bottle. As shown in Table 3 provided below, it was confirmed that Example 6 is excellent in fastness.

The assessment was conducted on the basis of the following criteria.

A. The water was as clear as the deionized water before immersion of the dyed tress thereinto, and the opposite side of the container was clearly seen.

B. Although the water was colored, the colored water did not hinder viewing of the opposite side of the container.

C. The water was colored, and the opposite side of the container was slightly invisible.

D. The color of the water was dark, and the opposite side of the container was almost invisible.

E. The color of the water was very dark, and the opposite side of the container was completely invisible.

TABLE 3

| | | Comparative | Comparative |
|---|---|---|---|
| | Example 6 | Example 3 | Example 4 |
| Dyeing fastness | B | A | D |

1. A hair cosmetic composition, which is a one-agent type hair cosmetic composition composed of a single agent, or a multi-agent type hair cosmetic composition composed of multiple agents,
wherein the hair cosmetic composition comprising the following components (A) to (D):
(A): one or more compounds selected from the group consisting of glyoxylic acid, a glyoxylic acid hydrate, a glyoxylate salt, and a glyoxylamide;
(B): one or more phenolic compounds selected from component (B1), (B2) and (B3):
(B1): resorcin;
(B2): a compound of formula (1) as defined below;
(B3): a compound of formula (2) as defined below;
(C): a direct dye; and
(D): water: 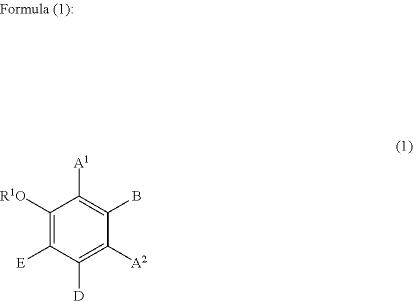
wherein:
R¹ represents a hydrogen atom or a methyl group;
A¹ and A¹, which may be identical to or different from each other, each represent a hydrogen atom, a C1 to C12 linear-chain or branched-chain alkyl or alkenyl group, an optionally substituted C7 to C12 aralkyl or arylalkenyl group, a C1 to C6 linear-chain or branched-chain alkoxy or alkenyloxy group, a halogen atom, or —CO—R¹ (wherein R¹ represents a C1 to C12 linear-chain or branched-chain alkyl or alkenyl group, an optionally substituted C7 to C12 aralkyl or arylalkenyl group, or an optionally substituted C6 to C12 aromatic hydrocarbon group);
B represents a hydrogen atom, a C1 to C12 linear-chain or branched-chain alkyl or alkenyl group, an optionally substituted C7 to C12 aralkyl or arylalkenyl group, or —OR³ or —COOR³ (wherein R³ represents a hydrogen atom or a C1 to C6 linear-chain or branched-chain alkyl or alkenyl group);
D represents a hydrogen atom, a hydroxyl group, a methyl group, or a C1 to C12 linear-chain or branched-chain alkoxy or alkenyloxy group; and
E represents a hydrogen atom, a hydroxyl group, a C1 to C6 linear-chain or branched-chain alkyl or alkenyl group, or a C1 to C6 linear-chain or branched-chain alkoxy or alkenyloxy group,
wherein two or three of A¹, A², B, and E each are a hydrogen atom, with each of remaining being a group which is not a sulfo group, and in the case where D is a hydrogen atom or a methyl group, A¹ and B, or A¹ and B form an optionally hydroxyl group-substituted benzene ring with two adjacent carbon atoms; 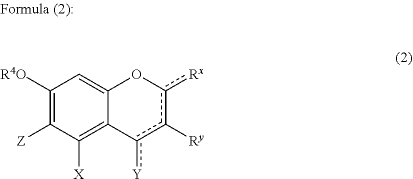
wherein,
R⁴ represents a hydrogen atom or a methyl group;
X represents a hydrogen atom, a hydroxyl group, or a methoxy group;
Y represents a hydrogen atom, an oxygen atom, a hydroxyl group, or a methoxy group;
Z represents a hydrogen atom or a C1 to C5 linear-chain or branched-chain alkyl or alkenyl group;
R^{X} represents a hydrogen atom, an oxygen atom, a hydroxy group, a methoxy group, or an aromatic hydrocarbon group, which is optionally substituted with up to three hydroxy groups or methoxy groups and which optionally forms a condensed ring with 1,3-dioxolane;
R^{y} represents a hydrogen atom, a hydroxy group, a methoxy group, or an aromatic hydrocarbon group, which is optionally substituted with up to three hydroxy groups or methoxy groups and which optionally forms a condensed ring with 1,3-dioxolane, or an arylcarbonyloxy group or aralkylcarbonyloxy group, which is optionally substituted with up to three hydroxy groups or methoxy groups;
a dotted line portion may be a double bond;
each of the combinations of the dotted line and the solid line being adjacent to R^{x} or Y denotes a double bond in the case where R^{x} or Y is an oxygen atom, and denotes a single bond in the other cases; and
Z represents a C1 to C5 linear-chain or branched-chain alkyl or alkenyl group, only when R^{x} or R^{y} is an o,p-dihydroxyaromatic hydrocarbon group, and represents a hydrogen atom in the other cases.

2. The hair cosmetic composition according to claim 1, wherein component (C) is one or more species selected from the group consisting of an anionic dye, a cationic dye, a nitro dye, HC Red 18, HC Blue 18, and HC Yellow 16, and salts of these species.

3. The hair cosmetic composition according to claim 1, wherein the molar ratio of component (B) to component (A), (B)/(A), of the hair cosmetic composition applied to hair is 0.001 or higher and lower than 2.5.

4. The hair cosmetic composition according to claim 1, which has a component (A) content, relative to the total composition of the hair cosmetic composition and in terms of glyoxylic acid, of 1 mass % or higher and 30 mass % or lower.

5. The hair cosmetic composition according to claim 1, which has a component (B) content, relative to the total composition of the hair cosmetic composition of 0.2 mass % or higher and 40 mass % or lower.

6. The hair cosmetic composition according to claim 1, wherein the glyoxylamide of component (A) is N-glyoxyloylcarbocysteine or N-glyoxyloylkeratinamino acid.

7. The hair cosmetic composition according to claim 1, wherein component (B) is component (B2).

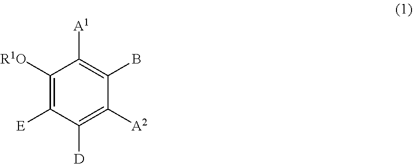

8. The hair cosmetic composition according to claim 1, wherein component (B) is component (B3).

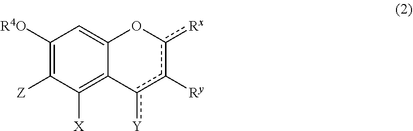

9. The hair cosmetic composition according to claim 1, wherein component (B) is component (B1), which is resorcin.

10. The hair cosmetic composition according to claim 1, which has a component (C) content, relative to the total hair cosmetic composition, of 0.001 mass % or higher and 10 mass % or lower.

11. The hair cosmetic composition according to claim 1, wherein the total amount of component reducing hair proteins, relative to the total hair cosmetic composition, is lower than 0.1 mass %.

12. The hair cosmetic composition according to claim 1, which is a one-agent type hair cosmetic composition.

13. The hair cosmetic composition according to claim 12, which has a pH of 4.0 or lower.

14. The hair cosmetic composition according to claim 1, which is a multi-agent type composition comprising a first agent containing components (B) and (D), and a second agent containing components (A) and (D), wherein component (C) is contained in the first agent and/or the second agent.

15. The hair cosmetic composition according to claim 14, wherein the second agent has a pH of 4.0 or lower.

16. A method for the treatment of hair, comprising the following steps (i) and (ii);
(i) a step of applying the hair cosmetic composition as recited in claim 12 to hair; and
(ii) a step of heating and shaping the hair to which the hair cosmetic composition is applied.

17. A method for the treatment of hair, comprising the following steps (i) and (ii);
(i) a step of mixing a first agent and a second agent of the hair cosmetic composition as recited in claim 14 and applying the obtained hair cosmetic composition to hair; and
(ii) a step of heating and shaping the hair to which the hair cosmetic composition is applied.

18. A method for the treatment of hair, comprising the following steps (i) and (ii);
(i) a step of applying a first agent of the hair cosmetic composition as recited in claim 14 to hair and then applying a second agent of the composition as recited in claim 14 onto the portion of hair where the first agent was applied; and
(ii) a step of heating and shaping the hair to which the hair cosmetic composition is applied.

19. The hair treatment method according to claim 18, which method further includes, after application of the first agent in step (i), and before application of the second agent to a portion of the hair to which the first agent has been applied, a step of rinsing the first agent applied onto the hair.

20. The hair treatment method according to claim 19, which method further includes, after application of the first agent in step (i), and before rinsing the first agent, a step of allowing the hair to stand for 1 minute or more and 1 hour or less.

21. The hair treatment method according to claim 16, which method further includes, after step (i), a step of allowing the hair to stand to which the hair cosmetic composition is to be applied, for 1 minute or more and 1 hour or less.

22. The hair treatment method according to claim 16, which method further includes, before step (i), a step of wetting hair.

23. The hair treatment method according to claim 16, wherein the heating temperature in step (ii) is 50° C. or higher and 250° C. or lower.

24. The hair treatment method according to claim 16, wherein step (ii) is performed under the conditions where evaporation of water is suppressed.

25. The hair treatment method according to claim 16, which method includes no step of applying, to the hair, a hair cosmetic composition containing a reducing agent or a strongly alkaline hair treatment agent having a pH of 12 or higher and 14 or lower.

26. The hair treatment method according to claim 16, comprising a step of heating and re-deforming the shape of hair to different shape after the step (ii).

27. The hair cosmetic composition according to claim 14, wherein the first agent has a pH of 6.0 or lower.
